(19) United States
(12) Patent Application Publication
(10) Pub. No.: US 2010/0204467 A1
(43) Pub. Date: Aug. 12, 2010

(54) LANTHANIDE (III) ION COMPLEXING COMPOUNDS, LUMINESCENT LANTHANIDE (III) ION COMPLEXES AND USE THEREOF AS FLUORESCENT LABELS

(75) Inventors: Laurent Lamarque, Saint Victor La Coste (FR); Craig Montgomery, West Yorkshire Yorkshire (GB); David Parker, Durham City Durham (GB)

Correspondence Address: MILLEN, WHITE, ZELANO & BRANIGAN, P.C., 2200 CLARENDON BLVD., SUITE 1400, ARLINGTON, VA, 22201, US

(73) Assignee: CIS BIO INTERNATIONAL, GIF-SUR-YVETTE CEDEX (FR)

(21) Appl. No.: 12/669,344

(22) PCT Filed: Jul. 18, 2008
(86) PCT No.: PCT/EP08/59444

§ 371 Date: Apr. 20, 2010

(30) Foreign Application Priority Data
Jul. 18, 2007 (GB) 0713963.7

(51) Int. Cl. C07D 225/02; C07F 15/00

(52) U.S. Cl. 540/483; 546/10

(57) ABSTRACT

Lanthanide (III) ion complexing compound comprising:

(1) a sensitizer moiety of formula (I)

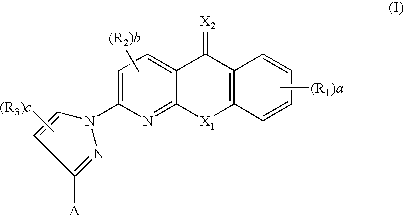

in which:
a is an integer from 1 to 4;
b is an integer equal 1 or 2;
c is an integer equal to 1 or 2;

(R₁)a, (R₂)b, (R₃)c are the same or different and are chosen from the group consisting of: H; alkyl; —COOR₄ where R₄ is H or an alkyl; aryl; heteroaryl; saturated or unsaturated cyclic hydrocarbon; CF3; CN; a halogen atom; L-Rg; L-Sc; or two consecutive R₃, two consecutive R₂ or two consecutive R₁ groups together form an aryl or a heteroaryl group or a saturated or unsaturated cyclic hydrocarbon group; where L is a linker, Rg is a reactive group and Sc is a conjugated substance;
X₁ and X₂ are the same or different and are O or S;
A is either a direct bond or a divalent group chosen from —CH₂— or —(CH₂)₂—, said moiety being covalently attached to
(2) a lanthanide (III) ion chelating moiety through A.

Application: Preparation of luminescent lanthanide (III) ion complexes and use thereof as fluorescent labels.

### FIELD OF THE INVENTION

This invention relates to novel compounds that can complex with lanthanide cations. In particular, this invention relates to complexing compounds which contain novel photosensitizers and can produce long-lived fluorescence for use in time-resolved energy transfer fluorescence assays, especially bioassays.

### STATE OF THE ART

Traditional fluorescent labels of organic dyes such as fluoresceins and rhodamines have long been employed as bioanalytical tools in immunoassays. Coordination complexes of the lanthanide (III) ions are more recently developed fluorescence agents and have been found to possess properties which make them very suited as potential labels in the bioassay field. These complexes are capable of giving long-lived and longer wavelength fluorescent emissions upon excitation. Through time-delay measurements, they have demonstrated clear advantages over conventional fluorescent labels in terms of experiencing less quenching and background interference while exhibiting increased detection sensitivity. In addition to these advantages, many lanthanide (III) complexes have improved solubility properties and are able to efficiently transfer energy from their excited states to neighbouring acceptor molecules. As such, they are ideal agents for time-resolved fluorescence use, especially for developing high-throughput automated and miniaturized binding assays with the inclusion of immunoassays, DNA hybridization assays, receptor binding assays, enzyme assays, cell-based assays, immunocytochemical or immunohistochemical assays.

Emissive lanthanide complexes that can be sensitised efficiently have been studied in detail as components of bioassays, spatially localised sensors, or as donors in time-resolved energy transfer systems. They typically comprise a polydentate ligand, often loosely termed a chelating moiety which binds the Lanthanide (III) ion and an organic sensitiser group. The sensitiser group has the function of absorbing light and transferring energy to the lanthanide. It thereby overcomes the inherently low absorbance of the lanthanide ions. There is a developing need to find long-lived emissive probes that are suitable for application in living cells (for recent examples: J. Yu, D. Parker, R. Poole, R. Pal and M. J. Cann, J. Am. Chem, Soc., 2006, 128, 2294; K. Hanoaka, K. Kikuchi, H. Kojima, Y. Urano and T. Nagano, J. Am. Chem. Soc., 2004, 126, 12470; G. Bobba, J-C. Frias and D. Parker, Chem. Commun., 2002, 890; H. C. Manning, S. M. Smith, M. Sexton, S. Haviland, M. F. Bai, K. Cederquist, N. Stella and D. J. Bornhop, Bioconjug. Chem., 2006, 17, 735; D. Parker and R. Pal, Chem. Commun., 2007, 474; H. C. Manning, T. Goebel, R. C. Thompson, R. R. Price, H. Lee and D. J. Bornhop, Bioconjug. Chem., 2004, 15, 1488; J-C. Frias, G. Bobba, M. J. Cann, D. Parker and C. J. Hutchinson, Org. Biomol. Chem., 2003, 1, 905). For such applications, the complexes need to be non-toxic and cell permeable, resistant to photobleaching and photo-fading, exhibit kinetic stability with respect to degradation and preferably should be relatively immune to quenching of the excited state of the lanthanide (III) ion by electron or charge transfer processes.

Several series of cyclic and acyclic ligands have been studied (e.g. R. Ziessel, N. Weibel, L. J. Charbonniere, M. Guardigli and A. Roda, J. Am. Chem. Soc., 2004, 126, 4888; B. Song, E. Wang and J. Yuan, Chem. Commun., 2005, 3553; M. Xiao and P. R. Selvin, J. Am. Chem. Soc., 2001, 123, 7067; D. Parker, R. S. Dickins, C. Crossland, J. A. K. Howard and H. Puschmann, Chem. Rev., 2002, 102, 1977) that present 8 or 9 donor atoms able to bind to the lanthanide ion and also incorporate a heterocyclic sensitising moiety that is able to harvest incident light efficiently (i.e. possess a large molar extinction coefficient, ε) and transfer its excited state energy in an intramolecular process to generate the lanthanide excited state. The ligand is preferably designed to inhibit the vibrational deactivation of the lanthanide (III) excited state, which can be particularly problematic with proximate OH and NH oscillators. (A. Beeby, I. M. Clarkson, R. S. Dickins, S. Faulkner, D. Parker, L. Royle, A. S. de Sousa J. A. G. Williams and M. Woods, J. Chem. Soc., Perkin Trans 2., 1999, 493). Recently, ligands containing substituted 1-azaxanthone and azathiaxanthones have been introduced (WO 2006/039505 A2; Org. Biomol. Chem., 2006, 4, 1707-1722; WO2006/120444 A1) as effective sensitisers for Eu and Tb emission in aerated aqueous media.

### DEFINITIONS

The term “alkyl” is used herein to refer to a branched or unbranched, saturated or unsaturated, monovalent hydrocarbon radical, generally having from about 1-15 carbons and preferably, from 1-10 carbons and more preferably from 1-6 carbons. Suitable alkyl radicals include, for example, structures containing one or more methylene, methine and/or methyne groups. Branched structures have a branching motif similar to i-propyl, t-butyl, i-butyl, 2-ethylpropyl, etc. As used herein, the term encompasses “substituted alkyls,” and “cyclic alkyls.”

“Substituted alkyl” refers to alkyl as just described including one or more substituents such as lower alkyl, aryl, acyl, halogen, hydroxy, amino, alkoxy, alkylamino, acylamino, thioamido, acyloxy, aryloxy, aryloxyalkyl, mercapto, thia, aza, oxo, both saturated and unsaturated cyclic hydrocarbons, heterocycles and the like. These groups may be attached to any carbon or substituent of the alkyl moiety. Additionally, these groups may be pendent from, or integral to, the alkyl chain.

“Alkylamino” refers to a secondary amine —NHR where R is an alkyl group as defined above.

“Alkylcarboxyl” refers to a group —RCOOH where R is an alkyl group as defined above.

The term “aryl” is used herein to refer to an aromatic substituent having 5 to 20 carbon atoms, preferably 5 to 10 carbon atoms; said aromatic substituent may be a single aromatic ring or multiple aromatic rings which are fused together, linked covalently, or linked to a common group such as a methylene or ethylene moiety. The common linking group may also be a carbonyl as in benzophenone. The aromatic ring(s) may include phenyl, naphthyl, biphenyl, diphenylmethyl and benzophenone among others. The term “aryl” encompasses “arylalkyl” and “substituted aryl.”

“Substituted aryl” refers to aryl as just described including one or more groups such as lower alkyl, acyl, halogen, haloalkyl (e.g. CF3), hydroxy, amino, alkoxy, alkylamino, acylamino, acyloxy, phenoxy, mercapto and both saturated or unsaturated cyclic hydrocarbons which are fused to the aromatic ring(s), linked covalently or linked to a common group such as a methylene or ethylene moiety. The linking group may also be a carbonyl such as in cyclohexyl phenyl ketone. The term “substituted aryl” encompasses “substituted arylalkyl.”

The term “arylalkyl” is used herein to refer to a subset of “aryl” in which the aryl group is attached to another group by an alkyl group as defined herein.

The term “Substituted arylalkyl” defines a subset of “substituted aryl” wherein the substituted aryl group is attached to another group by an alkyl group as defined herein.

The term “saturated cyclic hydrocarbon” denotes groups having 3 to 20 carbon atoms, preferably 3 to 12 carbon atoms, and more preferably 3 to 6 carbon atoms. Examples of these groups are cyclopropyl, cyclobutyl, cyclopentyl, etc., and substituted analogues of these structures. These cyclic hydrocarbons can be single-or multi-ring structures. The term “saturated cyclic hydrocarbon” encompasses “substituted saturated cyclic hydrocarbon”.

The term “substituted saturated cyclic hydrocarbon” refers to saturated cyclic hydrocarbon as just described including one or more groups such as lower alkyl, acyl, halogen, haloalkyl (e.g. CF3), hydroxy, amino, alkoxy, alkylamino, acylamino, acyloxy, phenoxy, mercapto, thia, aza, oxo.

The term “unsaturated cyclic hydrocarbon” is used to describe a monovalent non-aromatic group with at least one double bond and having 3 to 20 carbon atoms, preferably 3to 12 carbon atoms and more preferably 3 to 6 carbon atom, such as cyclopentane, cyclohexene, etc. and substituted analogues thereof. These cyclic hydrocarbons can be single-or multi-ring structures. The term “unsaturated cyclic hydrocarbon” encompasses “substituted unsaturated cyclic hydrocarbon”

The term “substituted unsaturated cyclic hydrocarbon ” refers to unsaturated cyclic hydrocarbon as just described including one or more groups such as lower alkyl, acyl, halogen, haloalkyl (e.g. CF3), hydroxy, amino, alkoxy, alkylamino, acylamino, acyloxy, phenoxy, mercapto, thia, aza, oxo.

The term “heteroaryl” as used herein refers to aromatic rings having 5 to 20 carbon atoms; preferably 5 to 10 carbon atoms and in which one or more carbon atoms of the aromatic ring(s) are replaced by a heteroatom such as nitrogen, oxygen or sulfur. Heteroaryl refers to structures that may be a single aromatic ring. multiple aromatic ring(s), or one or more aromatic rings coupled to one or more non-aromatic ring(s). In structures having multiple rings, the rings can be fused together, linked covalently, or linked to a common group such as a methylene or ethylene moiety. The common linking group may also be a carbonyl as in phenyl pyridyl ketone. As used herein, rings such as thiophene, pyridine, isoxazole, phthalimide, pyrazole, indole, furan, etc. or benzo-fused analogues of these rings are defined by the term “heteroaryl.” The term heteroaryl encompasses “substituted heteroaryl” and “heteroarylalkyl”

The term “Heteroarylalkyl” defines a subset of “heteroaryl” wherein an alkyl group, as defined herein, links the heteroaryl group to another group.

The term “substituted heteroaryl” refers to heteroaryl as described above wherein the heteroaryl nucleus is substituted with one or more groups such as lower alkyl, acyl, halogen, alkylhalos (e.g. CF₃), hydroxy, amino, alkoxy, alkylamino, acylamino, acyloxy, mercapto, etc. Thus, substituted analogues of heteroaromatic rings such as thiophene, pyridine, isoxazole, phthalimide, pyrazole, indole, furan, etc. or benzo-fused analogues of these rings are defined by the term “substituted heteroaryl” The term “substituted heteroaryl” encompasses “substituted heteroarylalkyl”.

The term “substituted heteroarylalkyl” refers to a subset of “substituted heteroaryl” as described above in which an alkyl group, as defined herein, links the heteroaryl group to another group.

The term “heterocyclic” is used herein to describe a monovalent saturated or unsaturated non-aromatic group having a single ring or multiple condensed rings from 1-12 carbon atoms and from 1-4 heteroatoms selected from nitrogen, sulfur or oxygen within the ring. Such heterocycles are, for example, tetrahydrofuran. morpholine, piperidine, pyrrolidine, etc.

The term “substituted heterocyclic” as used herein describes a subset of “heterocyclic” wherein the heterocycle nucleus is substituted with one or more groups such as lower alkyl, acyl, halogen, alkylhalos (e.g. CF3), hydroxy, amino, alkoxy, alkylamino, acylamino, acyloxy, mercapto, etc.

The term “heterocyclicalkyl” defines a subset of “heterocyclic” wherein an alkyl group, as defined herein, links the heterocyclic group to another group.

The term “halogen” is used herein to refer to fluorine, bromine, chlorine and iodine atoms.

The term “alkoxy” is used herein to refer to the —OR group, where R is alkyl, or a substituted analogue thereof. Suitable alkoxy radicals include, for example, methoxy, ethoxy, t-butoxy, etc.

The term “reactive group” is used to mean a first atom or group capable of reacting with a second atom or group forming a covalent bond with it.

The term “alkoxycarbonyl” by itself or as part of another substituent refers to a radical —C(O)OR where R represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

The term “amino acids side chain” refers to the following groups:

| Amino acid | Side chain |
|---|---|
| Alanine | —CH₃ |
| Cysteine | —CH₂SH |
| Aspartic acid | —CH₂COOH |
| Glutamic acid | —CH₂CH₂COOH |
| Phenylalanine | —CH₂C₆H₅ |
| Glycine | —H |
| Histidine | —CH₂—C₃H₃N₂ |
| Isoleucine | —CH(CH₃)CH₂CH₃ |
| Lysine | —(CH₂)₄NH₂ |
| Leucine | —CH₂CH(CH₃)₂ |
| Methionine | —CH₂CH₂SCH₃ |
| Asparagine | —CH₂CONH₂ |
| Proline | —CH₂CH₂CH₂— |
| Glutamine | —CH₂CH₂CONH₂ |
| Arginine | —(CH₂)₃NH—C(NH)NH₂ |
| Serine | —CH₂OH |
| Threonine | —CH(OH)CH₃ |
| Valine | —CH(CH₃)₂ |
| Tryptophan | —CH₂C₈H₆N |
| Tyrosine | —CH₂—C₆H₄OH |

### DESCRIPTION OF THE INVENTION

The invention relates to lanthanide (III) ion complexing compounds comprising: (1) a sensitizer moiety of formula (I)

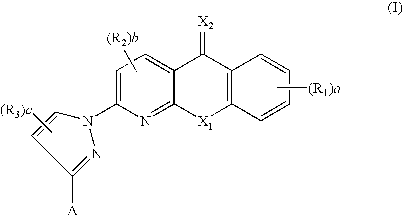

in which:
a is an integer from 1 to 4;
b is an integer equal 1 or 2;
c is an integer equal to 1 or 2;
(R₁)ₐ, (R₂)_{b}, (R₃)_{c} are the same or different and are chosen from the group consisting of H; alkyl; —COOR₄ where R₄ is H or an alkyl; aryl; heteroaryl; saturated or unsaturated cyclic hydrocarbon; CF3; CN; a halogen atom;
L-Rg; L-Sc; or two consecutive R₃, two consecutive R₂ or two consecutive R₁ groups together form an aryl or a heteroaryl group or a saturated or unsaturated cyclic hydrocarbon group;
where L is a linker, Rg is a reactive group and Sc is a conjugated substance;
X₁, X₂ are the same or different and are O or S;
A is either a direct bond or a divalent group chosen from: —CH₂— or —(CH₂)₂—, said moiety being covalently attached to
(2) a lanthanide (III) ion chelating moiety through A.

It should be specified that each R₁, R₂ and R₃ in the (R₁)ₐ groups, (R₂)_{b} groups and (R₃)_{c} groups, may be identical or different. For example, if a is 2, the two R₁ groups may be the same or different.

Particularly preferred compounds of formula I are:
those compounds where X₁═X₂═O;
those where a═b═c═1, R₂═R₃═H and R₁ is a (C₁-C₆) alkyl; and
those where a═b═c=1, X₁═X₂═O, R₂═R₃═H and R₁ is a (C₁-C₆) alkyl.

Other particularly preferred compounds of formula (I) are those in which:
a═b═c═1;
R₁═H, (C₁-C₆) alkyl;
R₂═H;
R₃═CF₃; COOR₄, where R₄═H, (C₁-C₆) alkyl, aryl, CN, halo, phenyl;
X₁═X₂═O.

### The Sensitising Moiety of Formula (I)

The pyrazoyl-azaxanthone sensitising moiety of formula (I) is also able to coordinate the lanthanide (III) ion via two nitrogen atoms of the pyrazoyl and azaxanthone groups. As compared to chelating compounds comprising azaxanthone chromophores disclosed in WO2006/120444 A1 and WO 2006/039505 A2, the addition of a pyrazoyl group extends the conjugation length of the chromophore, shifting the lowest energy absorption band of the lanthanide complex to longer wavelength and increasing the molar extinction coefficient.

The sensitising moiety of formula (I) is obtained by using a sensitising derivative of formula (Ia), which is a further object of the present invention:

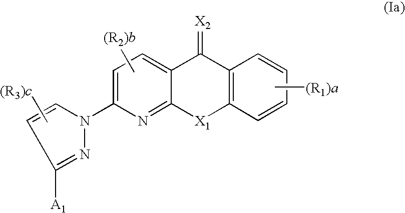

in which:
(R₁)ₐ, (R₂)_{b} and (R₃)_{c} are as defined hereinabove for moiety of formula (I);
A₁ is hydrogen, alkyl, halogen or halogenoalkyl.

Particularly preferred compounds of formula (Ia) are:
those in which X₁═X₂═O;
those where a═b═c═1, R₂═R₃═H and R₁ is a (C₁-C₆) alkyl; and
those where a═b═c═1 , X₁═X₂═O , R₂═R₃═H and R₁ is a (C₁-C₆) alkyl.

Other particularly preferred compounds of formula (Ia) are those in which:
a═b═c═1;
R₁═H, (C₁-C₆) alkyl;
R₂═H;
R₃═CF₃; COOR₄, where R₄═H, (C₁-C₆) alkyl, aryl, CN, halo, phenyl;
X₁═X₂═O.

The sensitising derivative of formula (Ia) is prepared by reacting a pyrazole derivative with a halo (preferably chloro) azaxanthone derivative. This reaction is based on nucleophilic substitution that occurs between the secondary amine group of the pyrazole and the halogenoalkyl substituent of the azaxanthone. (See reaction scheme 1—compounds (6) to (7)).

Pyrazole derivatives bearing various substituents are commercially available and can be used to prepare compounds according to the invention where R₃ is other that a hydrogen.

Synthesis of the chloroazaxanthone derivative is carried out by reaction of the 2-chloronicotinic acid with the 4-tert-butylphenol in the presence of NaOMe in MeOH according to the reaction scheme 1. It would be obvious to the person skilled in the art that the other halo azaxanthone derivatives may be obtained by processes similar to the one of reaction scheme 1 (compounds (1) to (6)) as the reagents used are commercially available.

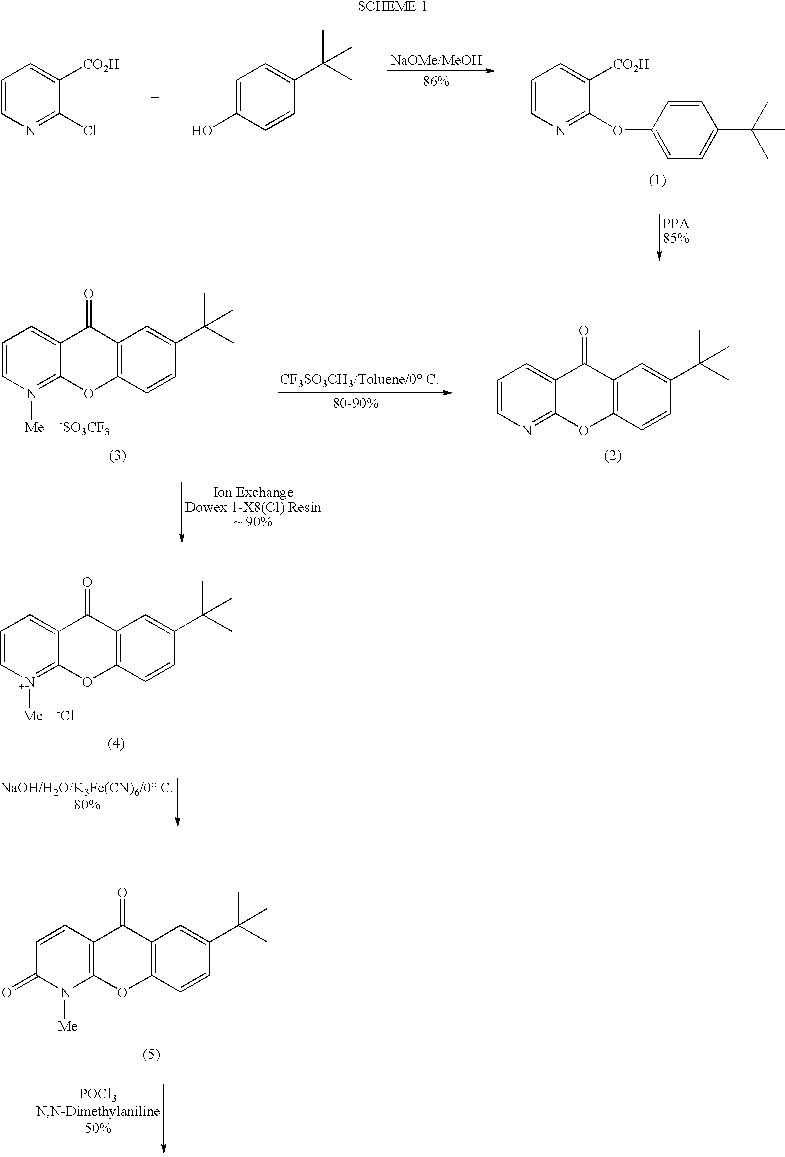

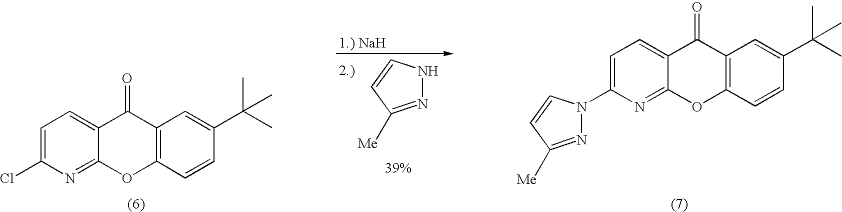

### The Lanthanide (III) Ion Chelating Moiety

The term “lanthanide (III) chelating moiety” is used to describe a group that is capable of forming a high affinity complex with lanthanide cations such as Tb³⁺, Eu³⁺, Sm³⁺, Dy³⁺. A lanthanide chelating moiety typically includes a set of lanthanide coordinating moieties that are heteroatom electron-donating group capable of coordinating a metal cation, such as O⁻, OPO₃²⁻, NHR, or OR where R is an aliphatic group. Such a lanthanide chelating moiety should be kinetically stable to exchange the lanthanide ion and preferably have a formation constant (K _{f}) of greater than 10¹⁰ M⁻¹.

A variety of useful chelating moieties are known to the person skilled in the art. Typical examples of lanthanide ion chelating moieties include: EDTA, DTPA, TTHA, DOTA, NTA, HDTA, DTPP, EDTP, HDTP, NTP, DOTP, DO3A, DOTAGA. Organic synthesis of these chelating moieties are known, and they are also available from commercial suppliers.

The following formulae illustrate chelating compounds that can be conjugated to a pyrazoyl-xanthone sensitizer and lead to the compounds according to the invention.

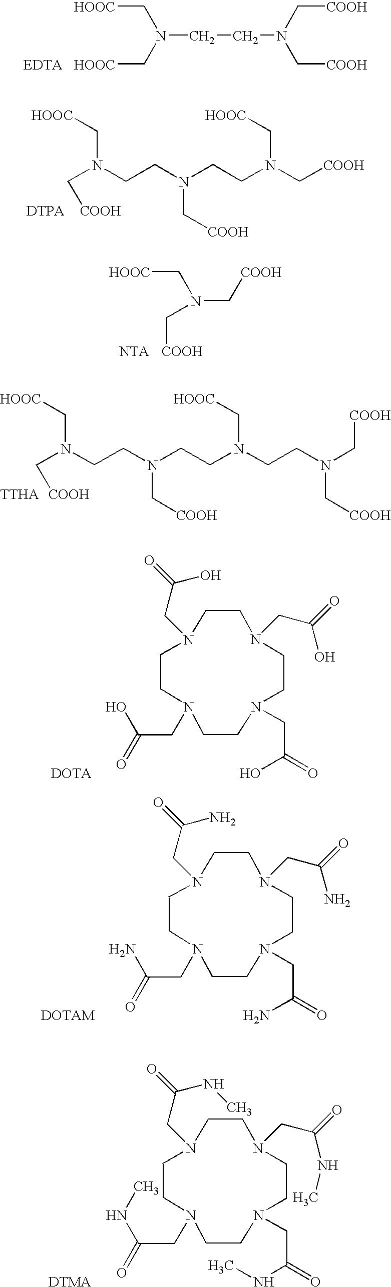

Most preferably, the sensitising moiety of formula (I) is linked to a lanthanide ion chelating and together form an ion complexing compound of formula (II):

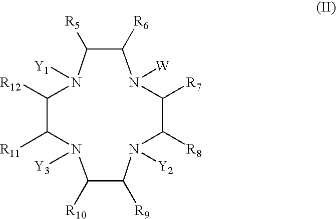

in which:
W is a sensitising moiety of formula (I) as defined above, linked through A,
R₅ to R₁₂ are the same or different and are chosen from the group consisting of: H, alkyl, L-Rg, L-Sc;
Y₁, Y₂ and Y₃ are the same or different and are chosen from the groups consisting of:
L-Rg, L-Sc, and groups of the following formulae:

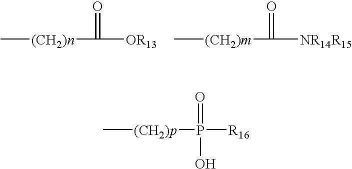

wherein:

n is 1 or 2;

m is 1 or 2;

p is 1 or 2;

R₁₃ is one of: H, lower alkyl, benzyl, L-Rg, L-Sc;

R₁₄, R₁₅ are the same or different and chosen from: H, —CHR′R″ in which R′ and R″ being the same or different and being chosen from: H, alkyl, optionally substituted aryl, optionally substituted aralkyl, or amino acid side chain, carboxyl group, L-Rg, L-Sc;

R₁₆ represents H, alkyl, optionally substituted aryl, preferably optionally substituted benzyl, lower alkylcarboxyl, lower alkylamino, L-Rg, L-Sc;

In a particular embodiment of the present invention, the lanthanide ion complexing compound is a compound of formula (III):

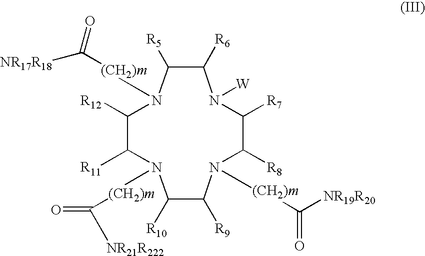

wherein:
W, R₅ to R₁₂ and m are as defined above,
R₁₇ to R₂₂ are the same or different and are chosen from: H, —CHR′R″ in which R′ and R″ being the same or different and being chosen from: H, alkyl, optionally substituted aryl, optionally substituted aralkyl, an amino acid side chain , a carboxyl group, L-Rg, L-Sc.

Among this family of compounds, a preferred subfamily comprises compounds of formula (IV):

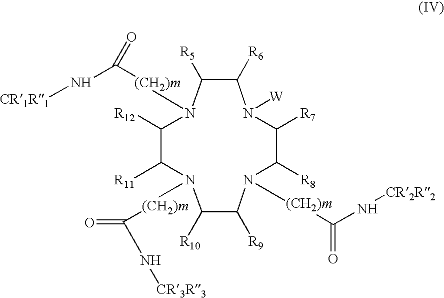

in which:
W, R₅ to R₁₂ and m are as defined above for a compound of formula (II);
R′₁, R′₂, R′₃ identical or different are a (C₁-C₆) alkyl, preferably —CH₃, —C₂H₅;
R″₁ to R″₃ are the same or different and are an optionally substituted aryl, preferably chosen from: optionally substituted benzyl, optionally substituted phenyl.

Another preferred subfamily comprises compounds of formula (V):

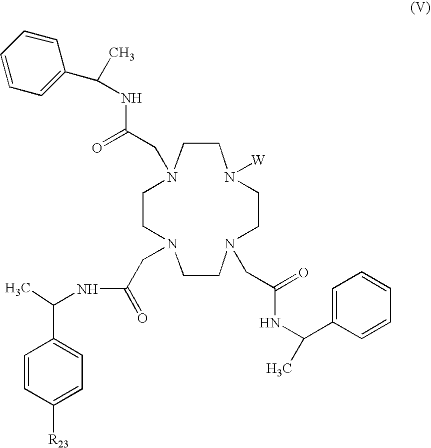

in which:
W is as previously defined for a compound of formula (II);
R₂₃ represents a carboxyl group, (C₁-C₆) alkoxycarbonyl, L-Sc, L-Rg.

In another embodiment of the present invention, the lanthanide ion chelating complex is a compound of formula (VI):

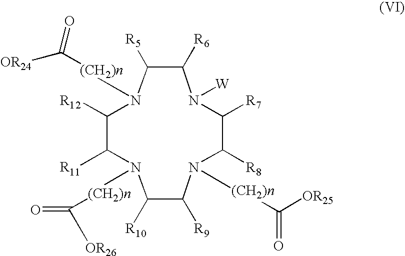

in which:
n is 1 or 2
W, R₅ to R₁₂ are as defined above for a compound of formula (II);
R₂₄ to R₂₆ are chosen from the group consisting of: H, (C₁-C₆) alkyl, optionally substituted aryl, (preferably optionally substituted benzyl), L-Rg, L-Sc.

In another embodiment, the lanthanide ion chelating complex is a compound of formula (VII):

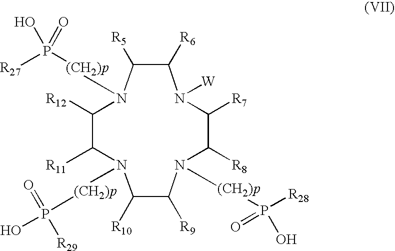

in which:
W, R₅ to R₁₂, and p are as previously defined for a compound of formula (II);
R₂₇ to R₂₉ are chosen from the group consisting of: H, (C₁-C₆) alkyl, optionally substituted aryl, (preferably optionally substituted benzyl), L-Rg, L-Sc.
   Any fluorescent lanthanide metal can be used with the chelating ligands of this invention but it is expected that complexes containing europium or terbium will possess the best fluorescent properties. Therefore, and most preferably, the lanthanide metal is terbium or europium.

### Linker-Reactive Group/Conjugated Substance: (L-Rg, L-Sc)

As mentioned above, compounds of formula (I) and (VII) optionally comprise a linker L that bears a reactive group Rg or a conjugated substance Sc. It is particularly advantageous to use the lanthanide ions complexes of the invention as fluorescent markers, particularly in bioassays where biological molecules have to be labelled with fluorescent compounds.

Some preferred compounds according to the invention comprise at least one group L-Rg or L-Sc, and preferably one or two.

The linker L is optionally a single covalent bond, such that either the reactive functional group Rg or the conjugated substance Sc is bound directly to the complexing compound. Alternatively, L may incorporate a series of non-hydrogen atoms that form a stable covalent linkage between the reactive functional group or conjugated substance and the lanthanide (III) ion complexing compound. Typically, L may incorporate 1-20 non-hydrogen atoms in a stable conformation. Stable atom conformations include, without limitation, carbon-carbon bonds, amide linkages, ester linkages, sulfonamide linkages, ether linkages, thioether linkages, and/or other covalent bonds. Preferred covalent linkages may include single bonds, carboxamides, sulfonamides, ethers, and carbon-carbon bonds, or combinations thereof.

Particularly preferred linkers are those according to the following formulae:

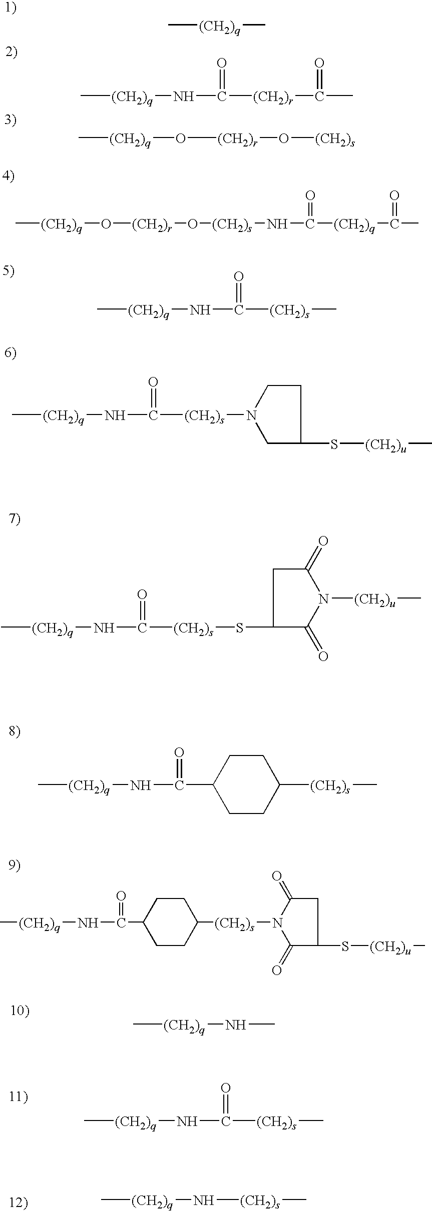

in which:
n and m are integers from 1 to 16, preferably 1 to 8;
p and r are integers from 1 to 16, preferably 1 to 5.

The reactive functional group Rg may include any functional group that exhibits appropriate reactivity to be conjugated with a desired substance. The choice of the reactive group depends on the functional groups present on the substance to be conjugated. Typically, functional groups present on such substances include, but are not limited to, alcohols, aldehydes, amines, carboxylic acids, halogens, ketones, phenols, phosphates, and thiols, or combinations thereof. Suitable Rg groups include activated esters of carboxylic acids, aldehydes, alkyl halides, amines, anhydrides, aryl halides, carboxylic acids, haloacetamides, halotriazines, hydrazines (including hydrazides), isocyanates, isothiocyanates, maleimides, phosphoramidites, sulfonyl halides and thiol groups, or a combination thereof. Typically, Rg is an activated ester of a carboxylic acid, an amino, haloacetamido, a hydrazine, an isothiocyanate, or a maleimide group. In one aspect of the lanthanide complex, Rg is a succinimidyl ester of a carboxylic acid.

Preferred reactive groups Rg are those that are routinely used in conjugation chemistry, and particularly those with following formulae:

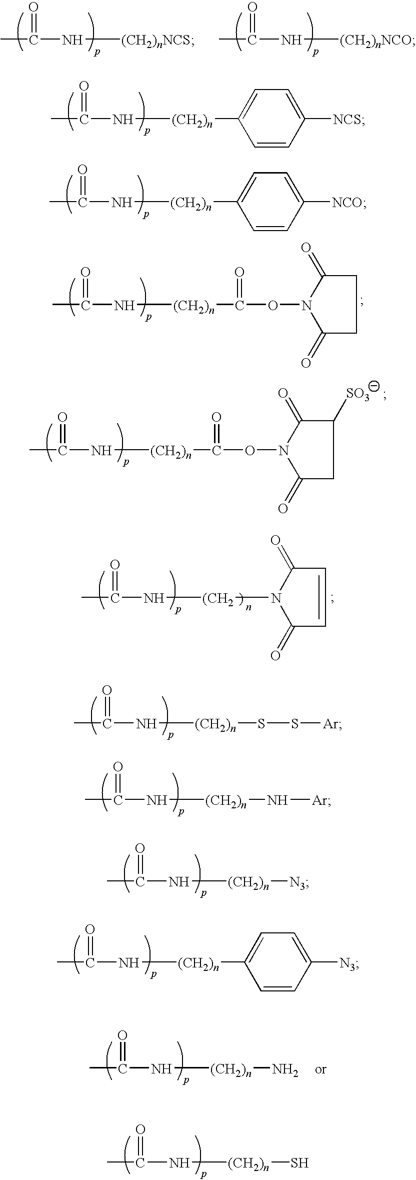

in which:
p represents 0 to 8 and n represents 0 or 1;
Ar is 5 to 6 member aryl, optionally containing 1 to 3 heteroatoms chosen from halo, N, O, S and optionally substituted by a halogen atom.

The lanthanide (III) ion complexing compounds are obtained by nucleophilic substitution resulting from the reaction of a sensitising derivative of formula (Ia) with a lanthanide (III) ion chelating derivative.

This process is illustrated by the following schemes 2 and 3. It would be obvious for the person skilled in the art that this process may be used starting from the reagents bearing the appropriate substituents.

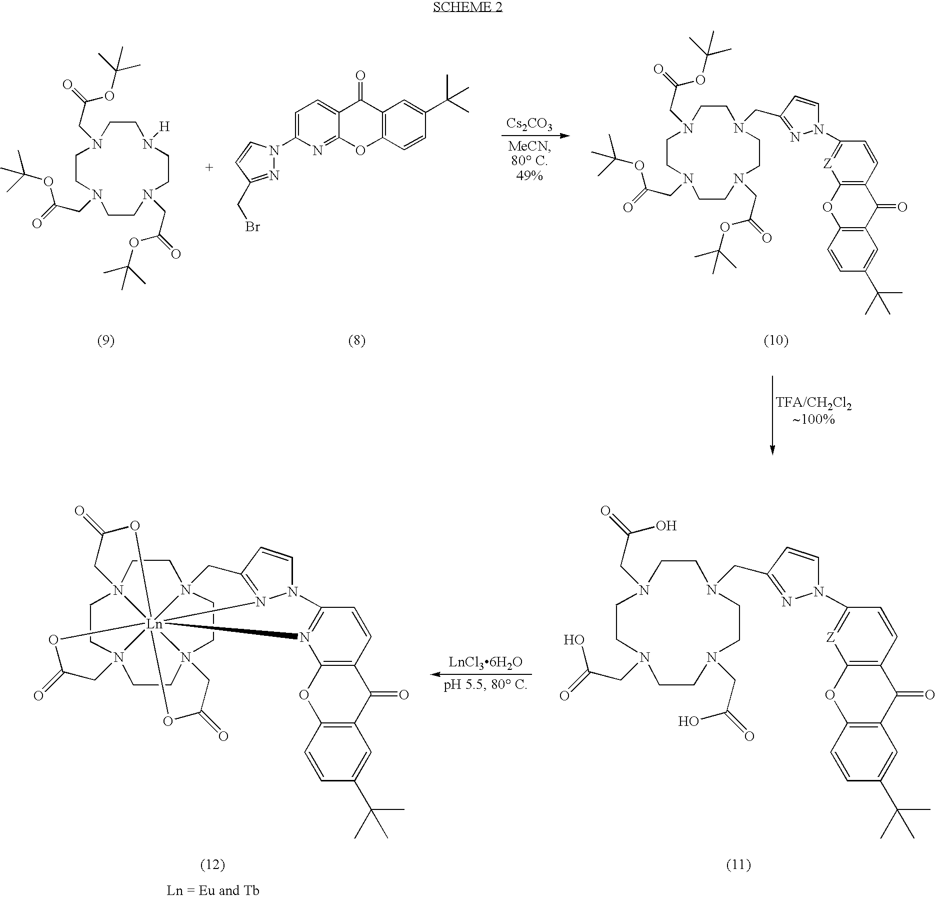

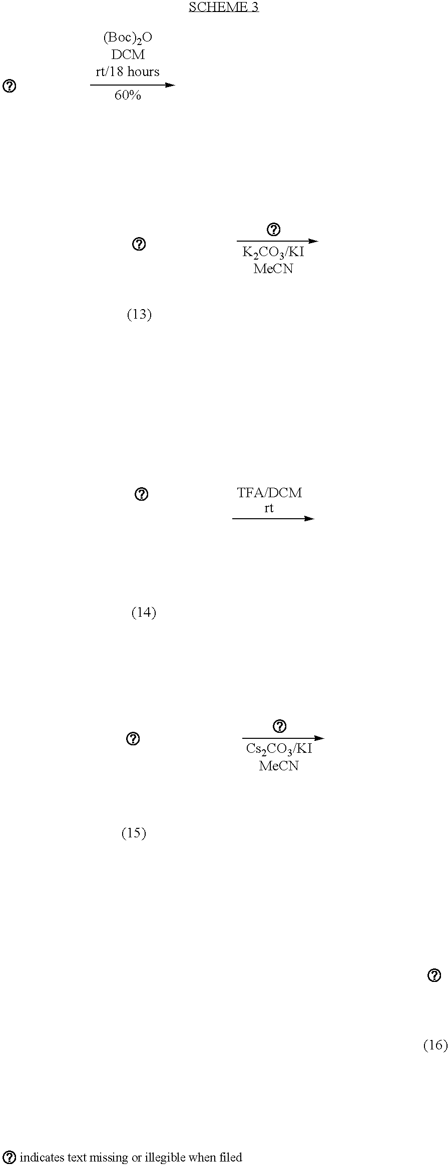

The lanthanide (III) ion complexing compounds that are substituted with a reactive functional group may be used to prepare a variety of conjugates. The conjugated substance may be a member of a specific binding pair. Alternatively, the conjugated substance may be a molecular carrier. The conjugated substance may include a biomolecule that is an amino acid, a peptide, a protein, a nucleoside, a nucleotide, an oligonucleotide, a nucleic acid polymer or a carbohydrate. The conjugated substance may include a polar moiety, or a masked polar moiety, or the conjugated substance may include a solid or semi-solid matrix. The conjugated substance may include one or more additional dyes or luminophores.

The conjugated substance Sc also may be a member of a specific binding pair or a molecular carrier. Specific binding pair members typically specifically bind to and are complementary with the complementary member of the specific binding pair. Conjugated members of a specific binding pair can be used to localize compounds of the present teachings to the complementary member of that specific binding pair. Representative specific binding pairs are: antigen/antibody, avidin or streptavidin/Biotin, ligand/receptor, DNA strand/DNA strand.

### Lanthanide (III) Ion Complexes

The invention also encompass those lanthanide (III) ion complexes obtained by contacting the lanthanide (III) ions complexing compounds of the invention and described hereinabove, with a lanthanide (III) ion (such as Tb³⁺, Eu³⁺, Sm³⁺, Dy³⁺). When the resulting complex is a charged compound, it is generally in the form of a salt with a counter ion, such as Cl^{-, OTf}⁻ or related common anions.

### EXAMPLES

Abbreviations used in the examples:

THF: Tetrahydrofuran

NBS: N-Bromosuccinimide

DO3A: 1,4,7-tris(carboxymethyl)-1,4,7,10-tetraazacyclododecane

DCM: Dichloromethane

TFA: Trifluoroacetic acid

OTf: Trifluoromethanesulfonate anion (═CF₃SO₃)

The invention will now be disclosed in more detail by the following illustrative, but non-limiting, examples 1 to 3 relating to the synthesis of the invention ligands and complexes and the Examples A to C concerning the properties of the invention complexes thus obtained.

### Example 1

### Synthesis of L¹ and [TbL¹] (See Schemes 1 and 2)

### 2-(4′-Tert-Butylphenoxy)Nicotinic Acid (1)

To a solution of sodium metal (1.02 g, 44.4 mmol) was carefully added to dry MeOH (25 cm³) was added 2-chloronicotinic acid (3.31 g, 21.01 mmol) and 4-tert-butylphenol (15.20 g, 101.18 mmol) to form a thick cream coloured solution. The MeOH was removed under reduced pressure to afford a cream residue which was heated for 20 h at 190 ° C. with stirring. After cooling, the coloured gum was treated with H₂O (200 cm³) and washed successively with Et₂O (2×150 cm³). The aqueous solution was acidified to pH 5 by the addition of acetic acid to afford a fine precipitate. The precipitate was filtered, washed with water and dried under vacuum to yield the title compound as a white fine crystalline solid (4.89 g, 18.02 mmol, 86%). δ_{H} (CDCl₃, 500 MHz) 1.37 (9H, s, ^{t}Bu), 7.14 (2H, d, J 8.5, H^{2′}), 7.20 (1H, dd, J 7.5; 5, H²), 7.49 (2H, d, J 9, H^{3′}), 8.35 (1H, dd, J 4.5; 2, H¹), 8.55 (1H, dd, J 8; 2, H³). δ_{C} (CDCl₃, 125 MHz) 31.7 (C^{6′}), 34.8 (C^{5′}), 113.5 (C⁴), 119.7 (C²), 121.4 (C^{2′}), 127.1 (C^{3′}), 143.8 (C³), 149.3 (C^{4′}), 149.8 (C^{1′}), 152.4 (C¹), 161.5 (C⁵), 164.9 (C═O_{(acid)}). m/z (ES⁻) 270.1 (100%, M—H). Found: C, 70.54; H, 6.20; N, 4.91%; C₁₆H₁₇NO₃ requires C, 70.83; H, 6.32; N, 5.16%.

### 7-Tert-Butyl-1-Azaxanthone (2)

Polyphosphoric acid (90 g) was added to 2-(4′-tert-butylphenoxy) nicotinic acid (2.15 g, 7.93 mmol) and the mixture heated at 120° C. for 16 h. The light brown mixture was allowed to cool slightly before being poured onto ice water (400 cm³) to afford a pale yellow solution. The pH of the solution was then adjusted to neutral pH 7 by the careful addition of concentrated NaOH(_{aq)}. The solution was extracted with Et₂O (3×300 cm³), the organic phases combined, dried over MgSO₄, filtered and the solvent removed under reduced pressure to afford 7-tert-butyl-1-azaxanthone as a cream coloured solid (1.79 g, 7.08 mmol, 89%). δ_{H} (CDCl₃, 500 MHz) 1.42 (9H, s, ^{t}Bu), 7.45 (1H, dd, J 7.5; 4.5, H²), 7.58 (1H, d, J 8.5, H¹⁰), 7.86 (1H, dd, J 9; 3, H⁹), 8.30 (1H, d, J 2.5, H⁷), 8.73-8.76 (2H, H¹/H³). δ_{C} (CDCl₃, 125 MHz) 31.6 (C¹⁴), 35.1 (C¹³), 117.0 (C⁴), 118.4 (C¹⁰), 121.1 (C²), 121.1, 122.7 (C⁷), 133.9 (C⁹), 137.6, 148.2 (C⁶), 154.1, 154.3(C¹²), 160.6 (C¹¹), 178.1 (C⁵). m/z (ES⁺) 529.5 (100%, 2M+Na), 782.3 (70%, 3M+Na), 275.8 (25%, M+Na). Found: C, 75.80; H, 5.91; N, 5.61%; C₁₆H₁₅NO₂requires C, 75.87; H, 5.97; N, 5.53%.

### 7-Tert-Butyl-N-methyl-1-Azaxanthonium Trifluoromethylsulfonate (3)

7-tert-Butyl-1-azaxanthone (1.00 g, 3.95 mmol) was dissolved in dry toluene (20 cm³) under an atmosphere of argon. The resultant yellow solution was then cooled in an ice bath to approximately 0° C. An excess of methyl trifluoromethanesulfonate (6 cm³, 8.70 g, 53.02 mmol) was then carefully added to the cooled solution in a dropwise fashion. Almost instantaneously a pale cream precipitate formed in a faint yellow coloured solute. The precipitate was filtered and dried under vacuum to afford the title compound as a white solid (1.49 g, 3.58 mmol, 91%). δ_{H} (CD₃OD, 400 MHz) 1.43 (9H, s, ^{t}Bu), 4.51 (3H, s, Me), 7.84 (1H, d, J 8.8, H¹⁰), 7.99 (1H, dd, J 8; 6, H²), 8.15 (1H, dd, J 8.8; 2.4, H⁹), 8.33 (1H, d, J 2.4, H⁷), 9.14 (1H, dd, J 6; 2, H¹), 9.30 (1H, dd, J 8; 2, H³). δ_{C} (CD₃OD, 100 MHz) 30.3 (C¹⁴), 34.8 (C¹³), 41.7 (CH₃), 118.2 (C¹⁰), 120.4 (C⁴), 120.8 (C⁶), 121.2 (C²), 122.6 (C⁷), 135.4 (C⁹), 145.9 (C³), 149.1 (C¹), 151.1 (C⁸), 152.4 (C¹¹), 156.3 (C¹²), 173.8 (C⁵). δ_{F} (CD₃OD, 188 MHz)−80.5 (CF₃). m/z (ES⁺) 268.2 (100%, M).

### 7-Tert-Butyl-N-Methyl-1-Azaxanthonium Chloride (4)

Compound (4), having the following properties, was obtained by ion exchange chromatography in water using a DOWEX 1-X8 (Cl) resin:

δ_{H} (CD₃OD, 500 MHz) 1.46 (9H, s, ^{t}Bu), 4.55 (3H, s, Me), 7.88 (1H, d, J 9, H¹⁰), 8.03 (1H, t, J 6.5, H²), 8.18 (1H, dd, J 9; 2, H⁹), 8.36 (1H, d, J 2, H⁷), 9.22 (1H, d, J 6.5, H¹), 9.33 (1H, d, J 7.5, H³). δ_{C} (CD₃OD, 125 MHz) 30.4 (C¹⁴), 34.8 (C¹³), 41.8 (CH₃), 118.2 (C¹⁰), 120.5 (C⁴), 120.8 (C⁶), 121.2 (C²), 122.6 (C⁷), 135.4 (C⁹), 145.9 (C³), 149.1 (C¹), 151.1 (C⁸), 152.4 (C¹¹), 156.3 (C¹²), 173.8 (C⁵).

### 6-Tert-Butyl-1-Methyl-1H-9-Oxa-1-Aza-Anthracene-2,10-Dione (5)

7-tert-Butyl-N-methyl-1-azaxanthonium chloride (0.36 g, 1.18 mmol) dissolved in H₂O (10 cm³) was added in a dropwise fashion to a solution of potassium hexacyanoferrate (III) (1.16 g, 3.54 mmol) in H₂O (6 cm³). The solution was cooled to approximately 0° C. and a solution of NaOH (0.85 g, 21.24 mmol) in H₂O (10 cm³) added to the reaction mixture over a period of 20 min. The solution was stirred at approximately 0° C. for 24 h. The solution was acidified to pH 3 by the addition of sulphuric acid to afford a green precipitate. The material was filtered, dissolved in CHCl₃ (50 cm³) and partitioned with H₂O (2×50 cm³). The organic phases were separated, dried over MgSO₄ and the solvent removed under reduced pressure to yield the title compound as a red solid (0.25 g, 0.87 mmol, 74%). δ_{H} (CDCl₃, 500 MHz) 1.41 (9H, s, ^{t}Bu), 3.76 (3H, s, Me), 6.54 (1H, d, J 9.5, H²), 7.47 (1H, d, J 8.5, H¹⁰), 7.79 (1H, dd, J 9; 2, H⁹), 8.21 (1H, d, J 9.5, H³), 8.29 (1H, d, J 2, H⁷). δ_{C} (CDCl₃, 125 MHz) 28.5 (CH₃), 31.6 (C¹⁴), 35.2 (C¹³), 102.8 (C⁴), 116.0 (C²), 117.3 (C¹⁰), 121.6 (C⁶), 122.9 (C⁷), 132.3 (C⁹), 135.7 (C³), 149.7 (C⁸), 152.0 (C¹¹), 156.5 (C¹²), 162.3 (C¹), 174.2 (C⁵). m/z (ES⁺) 284.3 (100%, M+H). HRMS (ES⁺) 284.12809; C₁₇H₁₈O₃N₁ requires 284.12812, [M+H]⁺. Found: C, 71.82; H, 5.91; N, 4.90%; C₁₇H₁₇NO₃ requires C, 72.07; H, 6.05; N, 4.94%.

### 7-Tert-Butyl-2-Chloro-1-Azaxanthone

N,N-Dimethylaniline (0.3 cm³) was added to a solution of 6-tert-butyl-1-methyl-1H-9-oxa-1-aza-anthracene-2,10-dione (0.18 g, 0.63 mmol) in POCl₃ (10 cm³) and the solution heated at reflux for 24 h. The solvent was removed under reduced pressure to yield a dark green residual solid. The residue was treated with H₂O (100 cm³) and the aqueous phase extracted with CH₂Cl₂ (2×50 cm³). The combined organic phases were washed with aqueous K₂CO₃ (0.1 M, 100 cm³), dried over K₂CO₃, filtered and the filtrate concentrated under reduced pressure. The residue purified by chromatography on silica (gradient elution: Hexane to 10% EtOAc/Hexane, R_{F}=0.33, 10% EtOAc/Hexane) to yield the title compound as a pink solid (0.09 g, 0.31 mmol, 49%). δ_{H} (CDCl₃, 500 MHz) 1.41 (9H, s, ^{t}Bu), 7.43 (1H, d, J 8, H²), 7.54 (1H, d, J 9, H¹⁰), 7.86 (1H, dd, J 9; 2.5, H⁹), 8.27 (1H, d, J 2.5, H⁷), 8.65 (1H, d, J 8, H³). δ_{C} (CDCl₃, 125 MHz) 31.5 (C¹⁴), 35.1 (C¹³), 115.6 (C⁴), 118.4 (C¹⁰), 121.1 (C⁶), 121.9 (C²), 122.7 (C⁷), 134.1 (C⁹), 139.9 (C³), 148.8 (C⁸), 153.8 (C¹¹), 155.6 (C¹²), 159.7 (C¹), 177.2 (C⁵).

### 7-Tert-Butyl-2-(N-3′-Methylpyrazole)-1-Azaxanthone (7)

Sodium hydride (30 mg, 1.25 mmol) was added to a solution of 3-methylpyrazole (88 mg, 1.07 mmol) in dry THF (5 cm³) under an atmosphere of argon. A solution of 7-tert-butyl-2-chloro-1-azaxanthone (280 mg, 0.97 mmol) in dry THF (5 cm³) was then added to the reaction mixture, which was stirred at 65° C. for 16 h. The reaction mixture was allowed to cool to room temperature before water (˜10 cm³) was added to the reaction mixture. The precipitate was collected via centrifugation and the resultant solid triturated with a minimum volume of Et₂O. The solvent was decanted to yield the title compound as a white solid (290 mg, 0.87 mmol, 90%). δ_{H} (CDCl₃, 400 MHz) 1.39 (9H, s, ^{t}Bu), 2.37 (1H, s, Me), 6.31 (1H, d, J 2.8, H^{2′}), 7.51 (1H, d, J 8.8, H¹⁰), 7.80 (1H, dd, J 8.8; 2.8, H⁹), 7.99 (1H, d, J 8.4, H²), 8.27 (1H, d, J 2.8, H⁷), 8.51 (1H, d, J 2.8, H^{1′}), 8.72 (1H, d, J 8.4, H³). δ_{C} (CDCl₃, 100 MHz) 14.2 (Me), 31.6 (C¹⁴), 35.1 (C¹³), 109.7 (C²), 110.1 (C^{2′}), 113.9 (C⁴), 118.1 (C¹⁰), 121.4 (C⁶), 122.7 (C⁷), 129.0 (C^{1′}), 133.4 (C⁹), 140.1 (C³), 148.3 (C⁸), 153.7 (C¹¹), 153.9 (C¹²), 154.0 (C^{3′}), 160.0 (C¹), 177.0 (C⁵). m/z (ES⁺) 688.9 (100%, 2M+H), 334.3 (50%, M+H). HRMS (ES⁺) 334.1551; C₂₀H₂₀O₂N₃ requires 334.1550, [M+H]⁺.

### 2-(N-3′-Bromomethylpyrazole)-7-Tert-Butyl-1-Azaxanthone (8)

N-Bromosuccinimide (NBS) (113 mg, 0.64 mmol) and dibenzoyl peroxide (10 mg, 0.04 mmol) were added to a solution of 7-tert-butyl-2-(1′-3′-methylpyrazole)-1-azaxanthone (212 mg, 0.64 mmol) in CCl₄ (15 cm³). The reaction mixture was heated at reflux under argon for 16 h. The reaction mixture was allowed to cool to room temperature, filtered and the solvent removed under reduced pressure to yield a yellow residue. The crude material was purified by chromatography on silica (100% CH₂Cl₂, RF=0.28, 100% CH₂Cl₂) to yield the title compound as a white solid (148 mg, 0.36 mmol, 56%). δ_{H} (CDCl₃, 500 MHz) 1.42 (9H, s, ^{t}B u), 4.56 (2H, s, CH₂Br), 6.62 (1H, d, J 2.5, H^{2′}), 7.56 (1H, d, J 9, H¹⁰), 7.85 (1H, dd, J 9; 2.5, H⁹), 8.06 (1H, d, J 8, H²), 8.31 (1H, d, J 2.5, H⁷), 8.63 (1H, d, J 2.5, H^{1′}), 8.80 (1H, d, J 8.5, H³). δ_{C} (CDCl₃, 125 MHz) 24.6 (CH₂Br), 31.6 (C¹⁴), 35.1 (C¹³), 109.7 (C^{2′}), 110.0 (C²), 114.6 (C⁴), 118.1 (C¹⁰), 121.4 (C⁶), 122.8 (C⁷), 129.8 (C^{1′}), 133.6 (C⁹), 140.5 (C³), 148.6 (C⁸), 153.3 (C¹¹), 153.7 (C^{3′}), 153.9 (C¹²), 159.9 (C¹), 177.0 (C⁵). m/z (ES⁺) 409.3 (100%, M+H), 846.6 (75%, 2M+H). HRMS (ES⁺) 434.04749; C₂₀H₁₈O₂N₃⁷⁹Br₁²³Na₁ requires 434.04746, [M+Na]⁺.

### 1, 4, 7-Tris(Tert-Butoxycarbonylmethyl)-1,4,7,10-Tetraazacyclododecane (9)

To a solution of tert-butyl bromoacetate (8.67 g, 44.23 mmol) in dry MeCN (75 cm³) was added cyclen (2.54 g, 14.70 mmol) and sodium hydrogen carbonate (3.72 g, 44.23 mmol). The reaction mixture was then stirred at room temperature under argon for 24 h. The solution was filtered and the filtrate solvent removed under reduced pressure to afford an orange residual oil, which crystallised upon standing. The crude material was purified by chromatography on silica gel (gradient elution: CH₂Cl₂: to 30% CH₂Cl₂/60% THF/5% MeOH/5% NH₃) to yield the title compound as a white crystalline solid (2.41 g, 4.68 mmol, 32%). δ_{H} (CDCl₃, 500 MHz) 1.47 (27H, s, ^{t}Bu), 2.88 (12H, br d, cyclen CH₂), 3.11 (4H, br s, cyclen CH₂), 3.30 (2H, s, acetate CH₂), 3.39 (4H, s, acetate CH₂), 10.04 (1H, br s, NH). δ_{C} (CDCl₃, 125 MHz) 28.4 (^{t}Bu), 30.6, 31.2, 47.8 (cyclen CH₂), 49.4 (acetate CH₂), 51.4 (cyclen CH₂), 58.5 (acetate CH₂), 81.9 (C^{t}Bu), 82.1(C^{t}Bu), 125.8, 169.9 (C═Oₑₛₜₑᵣ), 170.8 (C═Oₑₛₜₑᵣ). m/z (ES⁺) 515.6 (100%, M+H).

1-(7-Tert-Butyl-2-(N-Pyrazoylmethyl)-1-Azaxanthone)-4,7,10-Tris(Tert-Butoxycarbonylmethyl)-1,4,7,10-tetraazacyclododecane (10)

Caesium carbonate (50 mg, 0.153 mmol) was added to a solution of 1,4,7,10-tetraazacyclododecane1,4,7-triacetic acid (75 mg, 0.146 mmol) and 2-(3′-bromomethylpyrazole)-7-tert-butyl-1-azaxanthone (60 mg, 0.146 mmol) in dry MeCN (5 cm³). The reaction mixture was heated at reflux under argon for 16 h. The solvent was removed under reduced pressure and CH₂Cl₂ (10 cm³) added to the solid. The insoluble inorganic solid was removed by filtration and the filtrate concentrated under reduced pressure to yield yellow oil. The crude material was purified by column chromatography on silica (gradient elution; CH₂Cl₂ to 8% MeOH-92% CH₂Cl₂) to yield the title compound as a yellow oil (60 mg, 0.071 mmol, 49%). δ_{H} (CDCl₃, 500 MHz) 1.40 (H, s, tBu), 1.51 (H, s, tBu), 2.48-3.12 (22H, br m, Cyclen CH₂; 3×CH₂), 6.57 (1H, d, J 2.5, H^{2′}), 7.56 (1H, d, J 8.5, H¹⁰), 7.86 (1H, dd, J 9; 2.5, H⁹), 8.23 (1H, d, J 8.5, H²), 8.27 (1H, d, J 2.5, H⁷), 8.57 (1H, d, J 2.5, H^{1′}), 8.62 (1H, d, J 8.5, H³). δ_{C} (CDCl₃, 125 MHz) 28.3 (^{t}Bu), 31.5 (^{t}Bu), 35.1 (C^{t}Bu), 50.6, 51.8, 56.1 (CH₂), 56.7 (CH₂), 82.4 (C^{t}BU), 110.8 (C²), 110.9 (C^{2′}), 114.3, 118.2 (C¹⁰), 121.1, 122.7 (C⁷), 129.6 (C^{1′}), 133.9 (C⁹), 140.1 (C³), 148.7 (C⁸), 153.6(C¹¹), 153.9 (C^{1′}), 155.3 (C¹²), 159.8 (C¹), 172.9 (C═Oₑₛₜₑᵣ), 176.9 (C⁵) m/z(ES⁺) 846.5 (100%, M+H).

### Synthesis of Ligand L¹:1-(7-Tert-Butyl-2-(Pyrazoylmethyl)-1-Azaxanthone)-4,7,10-Tris(Carboxymethyl)-1,4,7,10-Tetraazacyclododecane (11)

A solution of 1-(7-tert-butyl-2-(methylpyrazole)-1-azaxanthone)-4,7,10-tris(tert-butoxycarbonylmethyl)-1,4,7,10-tetraazacyclododecane (45 mg, 0.053 mmol) in TFA (2 cm³) and CH₂Cl₂ (2 cm³) was stirred under argon at room temperature for 24 h. The solvents were removed under reduced pressure. The residue was repeatedly (3×) dissolved in CH₂Cl₂ (5 cm³) and the solvent removed under reduced pressure to facilitate elimination of excess add and tert-butyl alcohol. This procedure yielded the hydrolysed ligand as a pale yellow solid that was used directly for complexation with lanthanide ions.

### TbDO3AAzaPyrazole7^{t}Bu: [TbL¹] (12)

TbCl₃.6H₂O (10 mg, 0.0276 mmol) was added to a solution of 1-(7-tert-butyl-2-(methylpyrazole)-1-azaxanthone)-4,7,10-tris(carboxymethyl)-1,4,7,10-tetraazacyclo-dodecane (17 mg, 0.0251 mmol) in water (1 cm³) and MeOH (1 cm³). The pH of the solution was adjusted to 5.4 by the addition of 1M KOH solution. The reaction mixture was stirred under argon at 80° C. for 5 h. The pH dropped to 3.4 during this period and was consequently adjusted back to pH 5 by addition of 1M KOH. The reaction mixture was stirred at 80° C. for a further 16 h. The reaction mixture was allowed to cool to room temperature and the MeOH removed under reduced pressure. The pH of the remaining aqueous solution was raised to 10.0 using dilute KOH solution. The suspension was centrifuged before removing the solid precipitate by filtration. The pH of the aqueous solution was reduced to pH 5.5 by the addition of HCl and the solution freeze dried to yield the terbium complex as a white solid. λₘₐₓ (H₂O)=348 nm, τ (H₂O) 2.24 ms.

### Example 2

### Synthesis of Ligand L^{2a} and Complex [TbL^{2a}] (See Scheme 3)

### 1,4,7-Tri-Tert-Butoxycarbonyl-1,4,7,10-Tetraazacydododecane (13)

A solution of di-tert-butyl dicarbonate (6.08 g, 27.86 mmol) in CH₂Cl₂ (100 ml) was added dropwise to a stirred solution of cyclen (2.00 g, 11.61 mmol) in CH₂Cl₂ (300 ml).

The mixture was stirred at room temperature for 18 h. The solvent was removed under reduced pressure to yield a transparent oil, which was purified by column chromatography on silica (gradient elution: 100% CH₂Cl₂-5% MeOH/CH₂Cl₂, R_{F}=0.29 10% MeOH/CH₂Cl₂) to afford the title compound as a white crystalline solid (3.08 g, 6.51 mmol, 56%). δ_{H} (CDCl₃, 300 MHz) 1.42 (18H, s, 3×^{t}Bu), 1.44 (9H, s, ^{t}Bu), 2.81 (4H, br s, cyclen 2×CH₂), 3.28 (8H, br s, cyclen 4×CH₂), 3.60 (4H, br s, cyclen 2×CH₂). δ_{C} (CDCl₃, 75 MHz) 28.9 (^{t}Bu), 29.0 (^{t}Bu), 46.1 (cyclen CH₂), 49.9 (cyclen CH₂), 51.2 (cyclen CH₂), 79.4 (C), 79.6 (C), 155.8 (C═O), 156.0 (C═O). MS (ES⁺) m/z473.4 (100%, [M+H]⁺).

### 1-(7-Tert-Butyl-2-(Pyrazoylmethyl)-1-Azaxanthone)-4,7,10-Tris(Tert-Butoxycarbonyl-1,4,7,10-Tetraazacyclododecane (14)

1,4,7-tris-tert-butoxycarbonyl-1,4,7,10-tetraazacyclododecane (45 mg, 0.109 mmol), 2-(3′-bromomethylpyrazole)-7-tert-butyl-1-azaxanthone (57 mg, 0.119 mmol), potassium carbonate (19 mg, 0.131 mmol) and a catalytic amount (2 mgs) of KI were dissolved in a mixture of CH₃CN and DCM (5 cm³, 1:1). The reaction mixture was heated at reflux under argon for 16 h. The mixture was allowed to cool to room temperature before the insoluble inorganic salts were removed by filtration. The filtrate was concentrated under reduced pressure to afford a residual oil, which was purified by chromatography on silica gel (gradient elution: CH₂Cl₂ to 3% CH₃OH/CH₂Cl₂) to yield the title compound as a pale yellow solid (75 mg, 0.093 mmol, 85%). δ_{H} (CDCl₃, 500 MHz) 1.44 (36H, br s, 3×^{t}Bu), 2.78 (4H, br s, Cyclen 2×CH₂), 3.39 (8H, br, Cyclen 4×CH₂), 3.59 (4H, s, Cyclen 2×CH₂), 3.89 (2H, s, CH₂—PyAza), 6.44 (1H, d, J 3, H^{2′}), 7.56 (1H, d, J 8.5, H¹⁰), 7.86 (1H, dd, J 9; 2.5, H⁹), 8.04 (1H, d, J 8.5, H²), 8.30 (1H, d, J 2.5, H⁷), 8.60 (1H, d, J 2, H^{1′}), 8.78 (1H, d, J 8.5, H³). (CDCl₃, 125 MHz) 28.7 (3×^{t}Bu), 31.5 (^{t}Bu), 35.1 (C^{t}Bu), 47.5, 47.9, 48.4, 50.1, 53.6, 54.0, 55.3 (All cyclen CH₂, CH₂—PyAza), 79.5, 109.9 (C²), 110.8 (C^{2′}), 114.2, 118.1 (C¹⁰), 121.4, 122.8 (C⁷), 129.0 (C^{1′}), 133.6 (C⁹), 140.3 (C³), 148.5, 152.7, 153.6, 153.9, 155.6, 155.9, 159.9, 177.0 (C⁵).

### 1-(7-Tert-Butyl-2-(Pyrazoylmethyl)-1-Azaxanthone)-1,4,7,10-Tetraazacyclododecane 15)

A solution of 1-(7-tert-butyl-2-(pyrazoylmethyl)-1-azaxanthone)-4,7,10-tris(tert-butoxycarbonyl-1,4,7,10-tetraazacyclododecane (110 mg, 0.137 mmol) in TFA (2 ml) and CH₂Cl₂ (2 ml) was stirred at room temperature for 24 h. The solvents were removed under reduced pressure and the resulting residue repeatedly (×3) dissolved in CH₂Cl₂ (5 ml) and concentrated under vacuum to facilitate elimination of excess acid and tert-butyl alcohol. The residue was finally taken into a 1 M KOH solution (5 ml) and extracted with CH₂Cl₂ (3×5 ml). The organic layer was dried over K₂CO₃, filtered and the filtrate concentrated under reduced pressure to yield the title compound as an orange solid (65 mg, 0.129 mmol, 94%). δ_{H} (CDCl₃, 200 MHz) 1.39 (9H, s, ^{t}Bu), 2.70 (4H, br s, cyclen 2×CH₂), 2.80 (8H, br s, cyclen 4×CH₂), 2.85 (4H, br s, cyclen 2×CH₂), 3.83 (2H, s, CH₂—PyAza), 6.51 (1H, d, J 3, H^{2′}), 7.54 (1H, d, J 8.5, H¹⁰), 7.82 (1H, dd, J 9; 2, H⁹), 8.04 (1H, d, J 8.5, H²), 8.28 (1H, d, J 2, H⁷), 8.58 (1H, d, J 2, H^{1′}), 8.74 (1H, d, J 8.5, H³).

### Synthesis of Ligand L^{2a}: 1-(7-Tert-Butyl-2-(Pyrazoylmethyl)-1-Azaxanthone)-4,7,10-Tris[(S)-1-(1-Phenyl)Ethylcarbamoylmethyl]-1,4,7,10-Tetraazacyclododecane (16)

1-(7-tert-Butyl-2-(pyrazoylmethyl)-1-azaxanthone)-4,7,10-tetraazacyclododecane (39.2 mg, 0.078 mmol), 2-chloro-N-[(S)-methylbenzyl]ethanamide (49.6 mg, 0.250 mmol), Cs₂CO₃ (81.1 mg, 0.25 mmol) and a catalytic amount of KI were dissolved in dry MeCN (5 ml) and heated at reflux under argon for 16 h.

The reaction mixture was allowed to cool to room temperature before removing the inorganic salts by syringe filtration. The filtrate was concentrated under reduced pressure and dried under vacuum to afford a glassy solid. The crude material was sonicated in Et₂O (15 ml) to yield a fine pale yellow precipitate which was isolated via centrifugation. The material was sonicated in Et₂O and centrifuged twice more to yield the title compound as a free flowing pale yellow solid (62.3 mg, 0.063 mmol, 81%). δ_{H} (CDCl₃, 500 MHz) 1.41 (9H, s, ^{t}Bu), 1.52 (9H, br, 3×Me), 2.48-3.12 (20H, br, m, cyclen 4×CH₂; 3×CH₂), 3.77 (2H, s, CH₂), 4.05 (2H, s, CH₂—PyAza), 5.01 (3H, m, 3×CH), 5.07 (1H, m, CH), 6.38 (1H, br s, H^{2′}), 7.03-7.19 (15H, br m, 3×Ph), 7.54 (1H, d, J 8, H¹⁰), 7.84 (1H, dd, J 8; 2, H⁹), 7.96 (1H, d, J 8, H²), 8.30 (1H, d, J 2.5, H⁷), 8.60 (1H, d, J 2, H^{1′}), 8.81 (1H, d, J 8, H³). m/z(ES⁺) 988.4 (100%, M+H).

### Synthesis of Complex [Tbl^{2a}]: TbPh₃PyAza^{t}Bu: [TbL²](CF₃SO₃)₃ (17)

A solution of 1-(7-tert-butyl-2-(pyrazoylmethyl)-1-azaxanthone)-4,7,10-tris[(S)-1-(1-phenyl)ethylcarbamoylmethyl]-1,4,7,10-tetraazacyclododecane (16 mg, 0.016 mmol) and Tb(OTf)₃ (11.7 mg, 0.019 mmol) in dry CH₃CN (1 cm³) was heated at reflux under argon for 16 h. The solution was then dropped onto Et₂O (˜20 cm³) to yield a solid precipitate. The solid was isolated by centrifugation and the solvent decanted. The solid was re-dissolved in CH₃CN and the process repeated to yield an off-white solid product (13 mg, 0.011 mmol, 69%). λₘₐₓ (H₂O)=348 nm, τ(H₂O) 2.00 ms.

This complex was converted to the more water soluble chloride salt by ion exchange chromatography in water using a DOWEX 1×8 200-400 MESH Cl resin.

### Example 3

### Synthesis of Ligand L^{2b} and Complex [TbL²]³⁺

### (SS)-1-(7-Tert-Butyl-2-(Pyrazoylmethyl)-1-Azaxanthone)-4,10-(1-(1-Phenyl)Ethyl-Carbamoylmethyl)-1,4,7,10-Tetrazacyclododecane (18)

N-((S)-1-Phenyl-ethyl)-2-(7-[((S)-1-phenyl-ethylcarbamoyl)-methyl]-1,4,7,10-tetraaza-cyclododec-1-yl)-acetamide (104 mg, 0.211 mmol), 2-(3′-bromomethylpyrazole)-7-tert-butyl-1-azaxanthone (87 mg, 0.211 mmol) and NaHCO₃ (20 mg, 0.231 mmol) were dissolved in dry MeCN (5 ml) and heated at 60° C. for 18 h.

The reaction mixture was allowed to cool to room temperature before removing the inorganic salts by syringe filtration. The filtrate was concentrated under reduced pressure to afford a crude solid. The crude material was purified by chromatography on neutral alumina (gradient elution: CH₂Cl₂ to 0.5% CH₃OH/CH₂Cl₂) to yield the title compound as a pale cream coloured solid (124 mg, 0.150 mmol, 71%). m.p. 152-154° C. δ_{H} (CDCl₃, 500 MHz) 1.40 (9H, s, ^{t}Bu), 1.47 (6H, d, J 7, 2×Me), 2.58 (4H, br s, cyclen 2×CH₂), 2.79 (4H, br s, cyclen 2×CH₂), 2.91 (8H, br s, cyclen 4×CH₂), 3.36 (4H, s, CH₂C(O)), 3.71 (2H, s, CH₂—PyAza), 5.07 (2H, q, J 14; 7.5, 2×CH), 6.31 (1H, d, J 2.5, H^{2′}), 7.13 (2H, t, J 7.5, Ph), 7.21 (4H, t, J 7.5, Ph), 7.35 (4H, d, J 7.5, Ph), 7.54 (1H, d, J 9, H¹⁰), 7.84 (1H, dd, J 8.5; 2.5, H⁹), 7.91 (2H, br s, 2×NH), 7.98 (1H, d, J 8.5, H²), 8.29 (1H, d, J 2.5, H⁷), 8.55 (1H, d, J 2.5, H^{1′}), 8.76 (1H, d, J 8.5, H³). δ_{C} (CDCl₃, 125 MHz) 15.5, 22.2 (2C, Me), 31.6 (3C, ^{t}Bu), 35.1, 46.5, 49.4 (2C, CH), 50.9 (cyclen CH₂), 52.3 (cyclen CH₂), 52.7 (Cyclen CH₂), 60.6 (2C, CH₂CO), 66.1, 110.0, 111.0, 114.4, 118.1, 121.3, 122.8, 126.0, 126.8, 127.0, 127.3, 128.5, 128.7, 128.8, 129.1, 133.7, 140.5, 144,0, 148.6, 153.3, 153.9, 160.0, 170.2, 177.0. MS (ES⁺) m/z 826.0 (100%, [M+H]⁺); HRMS (ES⁺) m/z found 826.47696 [M+H]⁺ C₄₈H₆₀N₉O₄ requires 826.47628.

Methyl 4-[(S)-1-(2-{7-[1-(6-Tert-Butyl-10-Oxo-10H-9-Oxa-1-Aza-Anthracen-2-yl)-1H-Pyrazol-3-ylmethyl]-4,10-Bis-[((S)-1-Phenyl-Ethylcarbamoyl)-Methyl]-1,4,7,10Tetraaza-Cyclododec-1-yl}-Acetylamino)-Ethyl]-Benzoate (19)

(S,S)-1-(7-tert-Butyl-2-(pyrazoylmethyl)-1-azaxanthone)-4,10-(1-(1-phenyl) ethylcarbamoylmethyl)-1,4,7,10-tetraazacyclododecane (60 mg, 0.073 mmol), methyl [N-2-(chloroethanoyl)-4-(S)-(1-aminoethyl)]benzoate (21.3 mg, 0.084 mmol) and Cs₂CO₃ (31 mg, 0.095 mmol) were dissolved in dry MeCN (3 ml) and heated at reflux under argon for 16 h.

The reaction mixture was allowed to cool to room temperature before removing the inorganic salts by syringe filtration. The filtrate was concentrated under reduced pressure and dried under vacuum to afford a glassy solid. The crude material was sonicated in Et₂O (10 ml) to yield a fine pale yellow precipitate which was isolated via centrifugation. The material was sonicated in Et₂O and centrifuged twice more to yield the title compound as a free flowing cream coloured solid (60 mg, 0.058 mmol, 79%). m.p. 187-189° C. δ_{H} (CDCl₃, 200 MHz) 1.41 (12H. s, ^{t}Bu; 1×Me), 1.45 (6H, d, J 7, 2×Me), 2.57 (16H, br, cyclen 8×CH₂), 2.86 (2H, br s, CH₂CO), 2,99 (4H, br s, 2×CH₂CO), 3.62-3.84 (5H, br, CH₂—PyAza; CO₂Me), 5.14 (3H, q, J 7, 3×CH), 6.30 (1H, d, J 2.5, H^{2′}), 6.21-7.32 (12H, br, 2×Ph; 2×Ar), 7.47 (2H, d, J 8, 2×Ar), 7.55 (1H, d, J 9, H¹⁰), 7.82 (1H, dd, J 8; 2.5, H⁹), 7.98 (1H, d, J 8, H²), 8.31 (1H, d, J 2.5, H⁷), 8.78 (1H, d, J 8, H³). MS (ES⁺) m/z 1067.7 (100%, [M+Na]; HRMS (ES⁺) m/z found 1045.5706 [M+H]⁺C₆₀H₇₃N₁₀O₇ requires 1045.5698 HPLC (t_{R}=12.36 min)

### Synthesis of Complex [TbL^{2b}]:TbPh₂PhCO₂MePyAza^{t}Bu: [TbL^{2b}]³⁺ (CF₃SO₃)₃ (20)

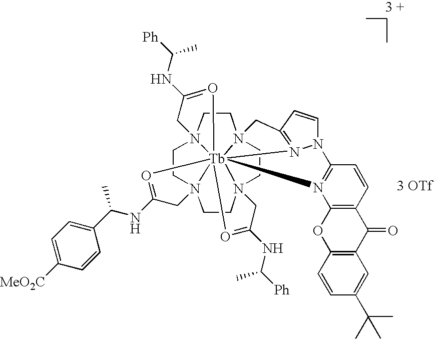

4-[(S)-1-(2-{7-[1-(6-tert-Butyl-10-oxo-10H-9-oxa-1-aza-anthracen-2-yl)-1H-pyrazol-3-ylmethyl]-4,10-bis-[((S)-1-phenyl-ethylcarbamoyl)methyl]-1,4,7,10tetraaza-cyclododec-1-yl}-acetylamino)-ethyl]-benzoic acid methyl ester (10 mg, 0.010 mmol) and Tb(OTf)₃ (6.7 mg, 0.011 mol) were dissolved in dry MeCN (1 cm³) and stirred at reflux under argon for 16 h. The solution was allowed to cool before being dropped onto Et₂O (˜25 cm³) to yield a solid precipitate. The solid was isolated by centrifugation and the solvent decanted. The solid was re-dissolved in CH₃CN and the process repeated to yield an off-white solid product (11.6 mg, 0.007 mmol, 74%). λₘₐₓ (H₂O)=348 nm, τ (H₂O)=2.27 ms. HPLC: t_{R}=9.81 min.

This complex was converted to the more water soluble chloride salt by ion exchange chromatography in water using a DOWEX 1×8 200-400 MESH Cl resin.

### Example A

### Spectral Properties of the Terbium(III) Complexes of L¹ and L²

The invention complexes obtained with the following ligands L¹, L^{2a} and L^{2b} according to the procedure as disclosed in the illustrative examples 1 to 3 above, were compared with the azaxanthone complexes [TbL³], [TbL³]³⁺and the related tetrazatriphenylene complexes [TbL⁵] and [TbL⁶]³⁺.

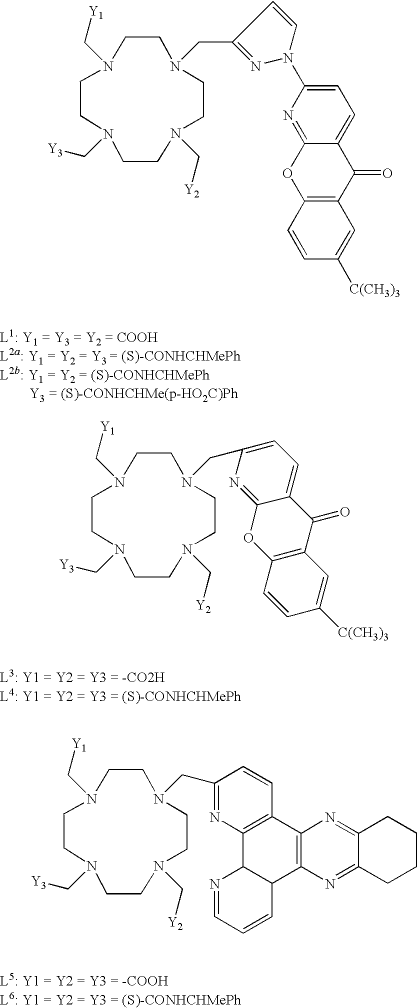

Absorption, emission and triplet energy data for these complexes are collated in Table 1.

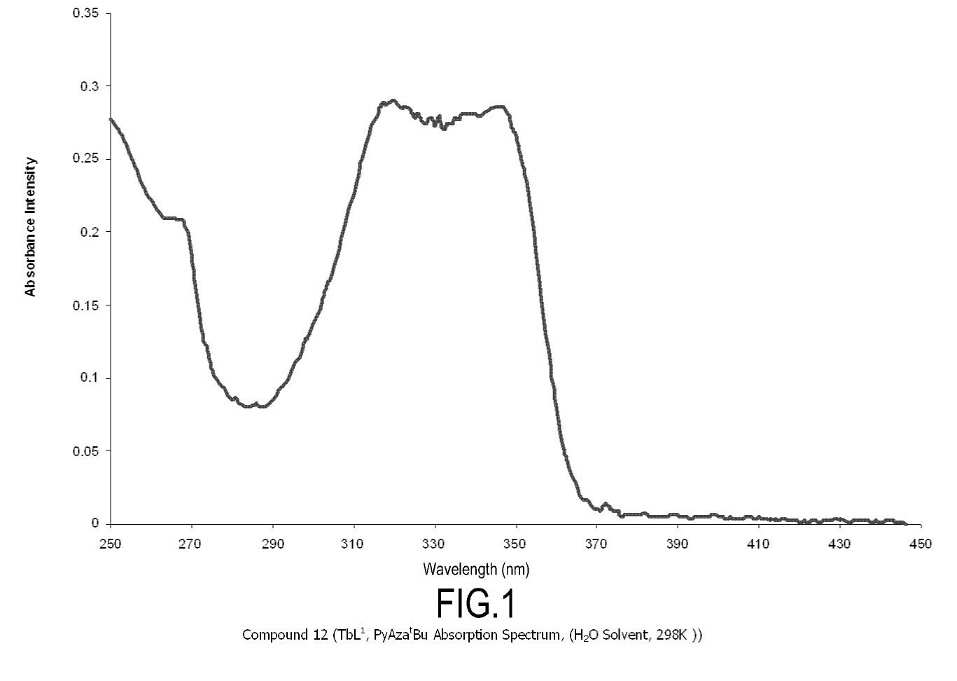

The absorption spectrum of [TbL¹] (FIG. 1) shows a fairly intense long wavelength band at 348 nm (ε 15,050M⁻¹ cm⁻¹) whose absorbance falls quite sharply, but still absorbs well at 355 nm, a common laser excitation wavelength.

The molar extinction coefficient for [TbL¹] is about twice that of the [TbL³]/[TbL⁵] analogues.

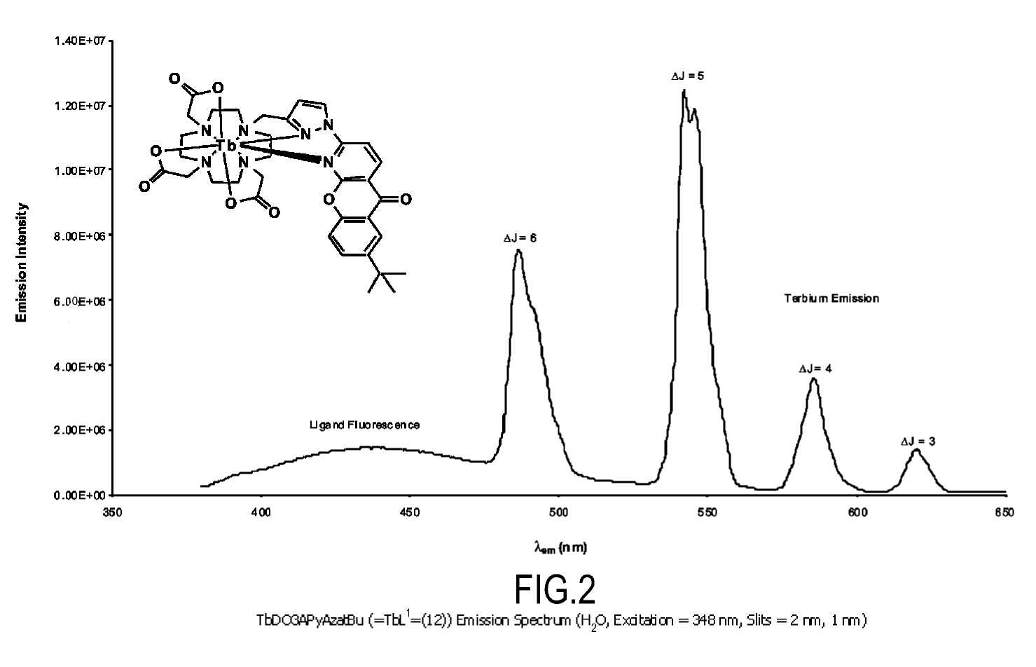

The total emission spectrum, (FIG. 2), reveals the expected Tb spectral fingerprint for decay of the ⁵D₄ excited state (τ_{H}_{₂}_{O} 2.24 ms for [Tb.L¹], and also shows some azaxanthone fluorescence (φₑₘ^{f} ˜15%) centred at 445 nm.

TABLE 1
Absorption, emission, lifetime (±10%) triplet energies (77K) and
quantum yields (±15%) data for Tb(III) complexes of L¹-L⁵

| | γₘₐₓ | ε | k_{D}_{₂}_{O}^{Tb} | k_{D}_{₂}_{O}^{Tb} | nₑₘ^{Tb} | E_{T}^{Gd} |
|---|---|---|---|---|---|---|
| Complex | (nm) | (M⁻¹ cm⁻¹) | (ms)⁻¹ | (ms)⁻¹ | (H₂O) | (cm⁻¹, 77K) a) |
| [TbL¹] | 348 | 15,050 | 0.44 | 0.37 | 15^{d} | 23,470 |
| [TbL²]³⁺ | 348 | 15,050 | 0.50 | 0.42 | 18^{d} | 23,470 |
| [TbL³] | 336 | 6,900 | 0.55 | 0.37 | 24 | 24,800 |
| [TbL⁴]³⁺ | 335 | 6,900 | 0.61 | 0.35 | 37 | 24,800 |
| [TbL⁵] | 348 | 8,300 | 0.68 | 0.60 | 33 | 23,800 |
| [TbL⁶]³⁺ | 348 | 8,300 | 0.64 | 0.58 | 40 | 23,800 |

a) triplet energies were measured on Gd analogues in a frozen glass of Et₂O/isopentane/ethanol or EtOH/MeOH (2:1).

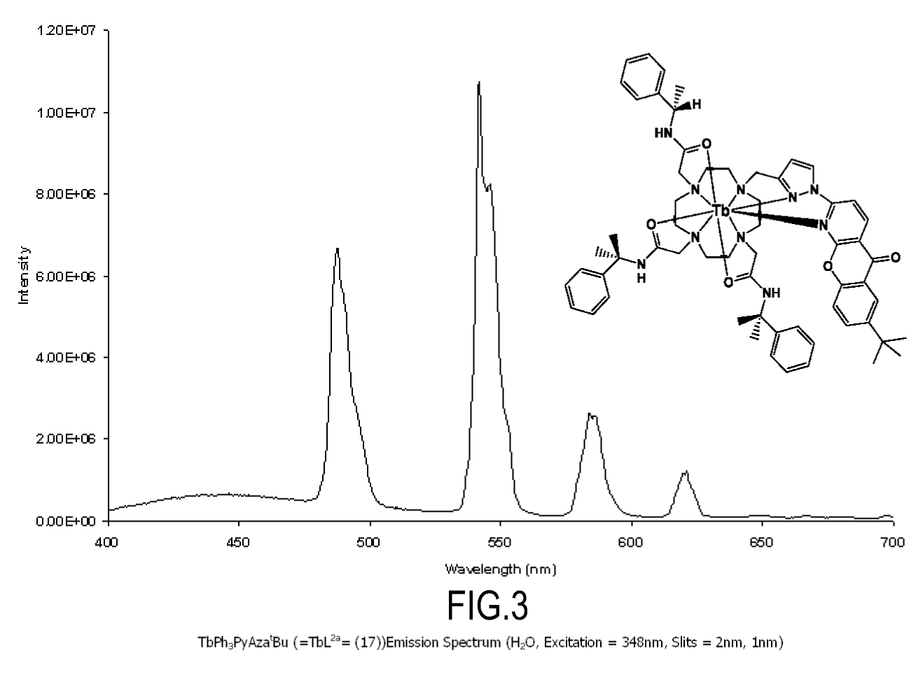

This ligand-based emission, whilst reducing the metal-based quantum yield, provides another observable emission band for luminescence microscopy and facilitates flow cytometric studies in cell-sorting/counting analyses. The total emission spectrum for [TbL²]³⁺ is shown in FIG. 3.

### Example B

### Quenching Studies with Terbium(III) Complexes of L¹ and L²

A key issue in assessing the utility of such luminescent complexes for intracellular application is their sensitivity towards quenching by endogenous electron-rich species (e.g. urate/ascorbate) either when the complexes are free or protein bound. Accordingly, the Stern-Volmer quenching constants (K_{Sv}⁻¹/mM) defining their relative sensitivity to quenching can be assessed comparatively, (Table 2).

TABLE 2
Sensitivity of terbium complexes of L¹ and L² to excited state quenching
by urate, ascorbate or iodide (pH 7.4, 0.1 M HEPES, 298K)

| Complex | Kₛᵥ⁻¹ (urate) | Kₛᵥ⁻¹ ascorbate | Kₛᵥ⁻¹ (I⁻) |
|---|---|---|---|
| [TbL¹] | 0.03 | 1.39 | 5.35 |
| [TbL²]³⁺ | 0.05 | 0.36 | 13.8 |
| [TbL³] | 0.012 | 0.57 | 53.5 |
| [TbL⁴]³⁺ | 0.04 | 0.37 | 9.2 |
| [TbL⁵] | 0.006 | 0.38 | 2.1 |
| [Tb L⁶]³⁺ | 0.025 | 0.18 | 0.9 |

The electron oxidation potential for ascorbate, iodide and urate follow the sequence: 0.30, 0,54 and 0.59 V (vs nhe) respectively.

The quenching process with urate is believed to involve an intermediate exciplex, involving a short-lived bonding π-π interaction. This is disfavoured as the sensitising chromophore becomes less susceptible to accept electron density and is reflected in the ligand reduction potential. For compound (7), this was −1.52 V (vs n.h.e., 298K, 0.1 M NBu₄ClO₄, MeCN), which compares to −1.07 V for the cyclohexyl tetraazatriphenylene and −1.60 V for the 1-aza-xanthone (2). Terbium complexes of L¹ and L² resisted urate and ascorbate quenching more effectively than the comparator complexes.

Protein binding of the Tb complex by serum albumin has also been shown to inhibit this process. Incremental addition of bovine serum albumin to [TbL²]³⁺ resulted in less than a 7% decrease in the terbium emission lifetime over the range 0.01 to 0.7 mM added protein. In the presence of 0.4 mM human serum albumin, 0.1 mM sodium urate and 0.2 mM ascorbate, (pH 7.4, 298K, 0.1M HEPES), the emission lifetime of [TbL²]³⁺ was within 10% of its value in water, i.e. 2.1 ms, compared to 0.7 ms and 0.5 ms for [TbL⁶]³⁺ and [TbL⁴]³⁺ respectively under the same conditions. Thus, protein association does not quench the excited state of [TbL²]³⁺, and the presence of protein shields the complex from quenching by two of the most common low MW reductants found in cells.

### Example C

### Preliminary Cell Microscopy Assessment

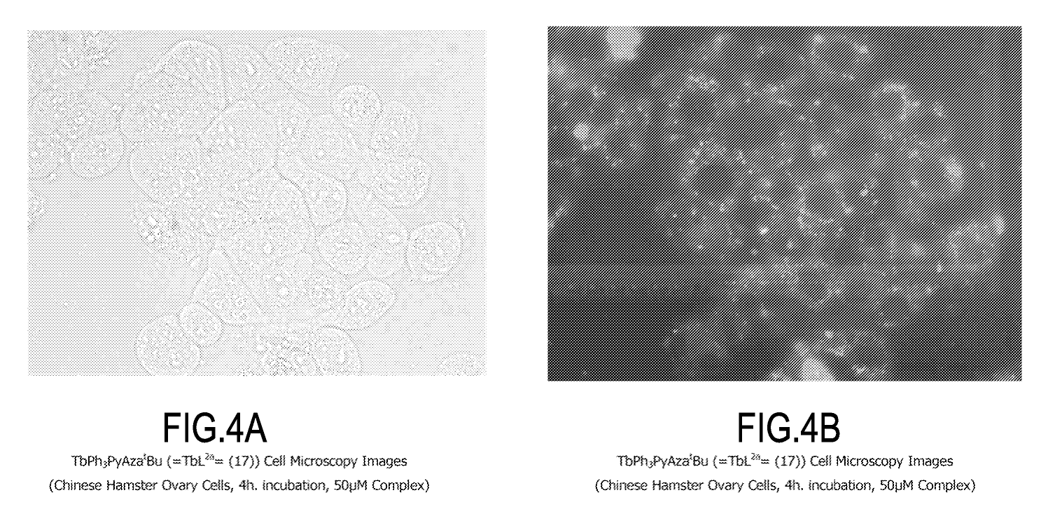

The complex [TbL²]³⁺ was incubated for 4 h (50 or 100 μM complex) with Chinese hamster ovarian cells, under standard conditions, that have been used previously to examine [TbL⁴]³⁺ and [TbL⁶]³⁺. Examination of the loaded cells by luminescence microscopy revealed complex uptake within the cell and localisation in the cytoplasm (FIGS. 4a and 4b) but without the tendency for nuclear localisation that has been observed with [TbL⁶]³⁺ and [TbL⁴]³⁺.

1. Lanthanide (III) ion complexing compound comprising:
(1) a sensitizer moiety of formula (I) 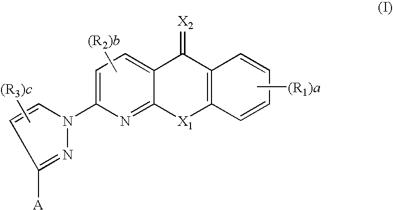
in which:
a is an integer from 1 to 4;
b is an integer equal 1 or 2;
c is an integer equal to 1 or 2;
(R₁)a, (R₂)b, (R₃)c are the same or different and are chosen from the group consisting of: H; alkyl; —COOR₄ where R₄ is H or an alkyl; aryl; heteroaryl; saturated or unsaturated cyclic hydrocarbon; CF3; CN; a halogen atom; L-Rg; L-Sc; or two consecutive R₃, two consecutive R₂ or two consecutive R₁ groups together form an aryl or a heteroaryl group or a saturated or unsaturated cyclic hydrocarbon group; where L is a linker, Rg is a reactive group and Sc is a conjugated substance;
X₁ and X₂ are the same or different and are O or S;
A is either a direct bond or a divalent group chosen from —CH₂— or —(CH₂)₂₋, said moiety being covalently attached to
(2) a lanthanide (III) ion chelating moiety through A.

2. Lanthanide (III) ion complexing compound according to claim 1 wherein X₁═X₂═O.

3. Lanthanide (III) ion complexing compound according to claim 1 wherein a═b═c=1, R2═R3═H and R1 is a (C₁-C₆) alkyl.

4. Lanthanide (III) ion complexing compound according to claim 1 wherein X1═X2═O, a═b═c═1, R2═R3═H and R1 is a (C₁-C₆) alkyl.

5. Lanthanide (III) ion complexing compound according to claim 1, wherein:
a═b═c═1;
R₁═H, (C₁-C₆) alkyl;
R₂═H;
R₃═CF₃; COOR₄, where R₄═H, (C₁-C₆) alkyl, aryl, CN, halo, phenyl;
X₁═X₂═O.

6. Lanthanide (III) ion complexing compound according to claim 1, wherein the chelating moiety comprises a set of heteroatom-containing electron-donating groups, such as carboxyl, amino, amido, oxo, alkylphosphinate or phosphonate.

7. Lanthanide (III) ion complexing compound according to claim 6, wherein it comprises 3, 4, 5, 6, 7, 8 or 9 heteroatom electron-donating groups.

8. Lanthanide (III) ion complexing compound according to claim 1, wherein the formation constant (K_{f}) of the lanthanide (III) ion complex is greater than 10¹⁰ M⁻¹.

9. Lanthanide (III) ion complexing compound according to claim 1, wherein the chelating moiety is chosen from the group consisting of: NTA, EDTA, DTPA, TTHA, a tetraazacyclododecane derivative such as DOTA.

10. Lanthanide (III) ion complexing compound according to claim 1, which is a compound of formula (II): 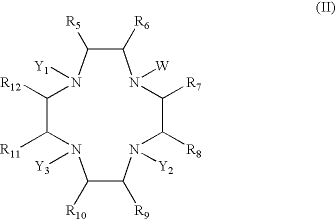
in which:
W is a sensitising moiety of formula (I) as defined in claim 1, linked through A,
R₅ to R₁₂ are the same or different and are chosen from the group consisting of H, alkyl, L-Rg, L-Sc;
Y₁, Y₂ and Y₃ are the same or different and are chosen from the groups consisting of L-Rg, L-Sc and groups of the following formulae: 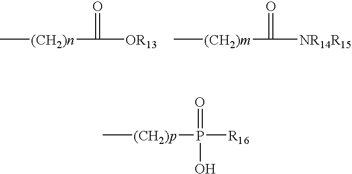
wherein:
n is 1 or 2;
m is 1 or 2;
p is 1 or 2;
R₁₃ is H, lower alkyl, benzyl, L-Rg, L-Sc;
R₁₄, R₁₅ are the same or different and chosen from H, —CHR′R″ in which R′ and R″ being the same or different and being chosen from H, alkyl, optionally substituted aryl, optionally substituted aralkyl, or amino acid side chain, carboxyl group, L-Rg, L-Sc;
R₁₆ represents H, alkyl, optionally substituted aryl, preferably optionally substituted benzyl, lower alkylcarboxyl, lower alkylamino, L-Rg, L-Sc.

11. Lanthanide (III) ion complexing compound according to claim 10, which is a compound of formula (III): 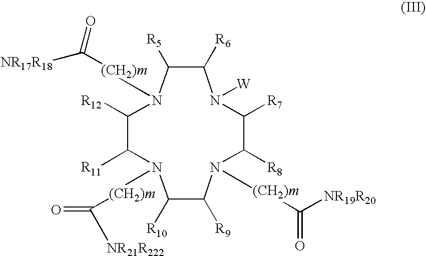
wherein
W, R₅ to R₁₂, m are as defined in claim 10,
R₁₇ to R₂₂ are the same or different and are chosen from H, —CHR′R″ in which R′ and R″ being the same or different and are chosen from H, (C₁-C₆) alkyl, optionally substituted aryl, optionally substituted aralkyl, amino acid side chain, carboxyl, L-Rg, L-Sc.

12. Lanthanide (III) ion complexing compound according to claim 11 which is a compound of formula (IV): 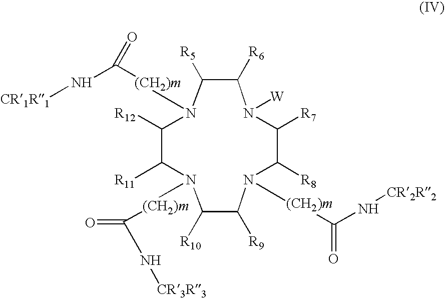
in which:
R′₁, R′₂, R′₃ are identical and represent a (C₁-C₆) alkyl, preferably —CH3, —C2H5;
R″₁ to R″₃ are the same or different and are an optionally substituted aryl, preferably optionally substituted benzyl or optionally substituted phenyl.

13. Lanthanide (III) ion complexing compound according to claim 12, which is a compound of formula (V): 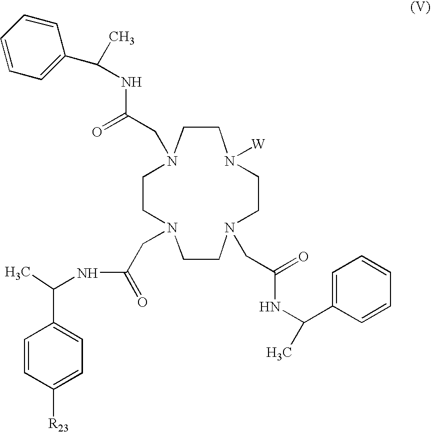
R₂₃ is H, a carboxyl group, a (C₁-C₆)alkoxycarbonyl, L-Sc, L-Rg

14. Lanthanide (III) ion complexing compound according to claim 10, which is a compound of formula (VI) 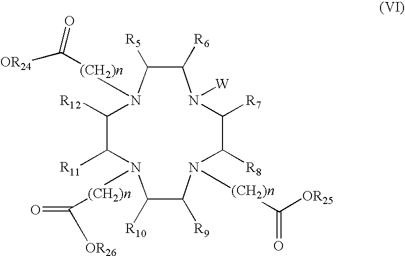
in which:
n is 1 or 2,
W, R₅-R₁₂, are as defined in claim 10,
R₂₄ to R₂₆ are chosen from the group consisting of H, (C₁-C₆) alkyl, optionally substituted aryl, (preferably optionally substituted benzyl), L-Rg, L-Sc H.

15. Lanthanide (III) ion complexing compound according to claim 10, which is a compound of formula (VII) 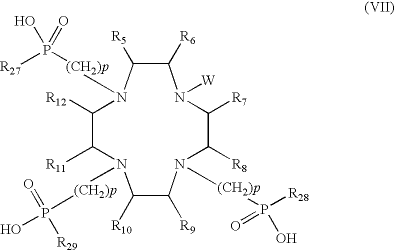
in which:
W, R₅-R₁₂, are as defined in claim 10,
R₂₇ to R₂₉ are chosen from the group consisting of: H, (C₁-C₆) alkyl, optionally substituted aryl, (preferably optionally substituted benzyl), L-Rg, L-Sc.

16. Lanthanide (III) ion complexing compound according to claim 1, wherein the linker L is a single covalent bond or L comprises 1-20 non-hydrogen atoms in a stable conformation such as carbon-carbon bonds, amide linkages, ester linkages, sulfonamide linkages, ether linkages, thioether linkages

17. Lanthanide (III) ion complexing compound according to claim 16, wherein L is chosen from the group consisting of: 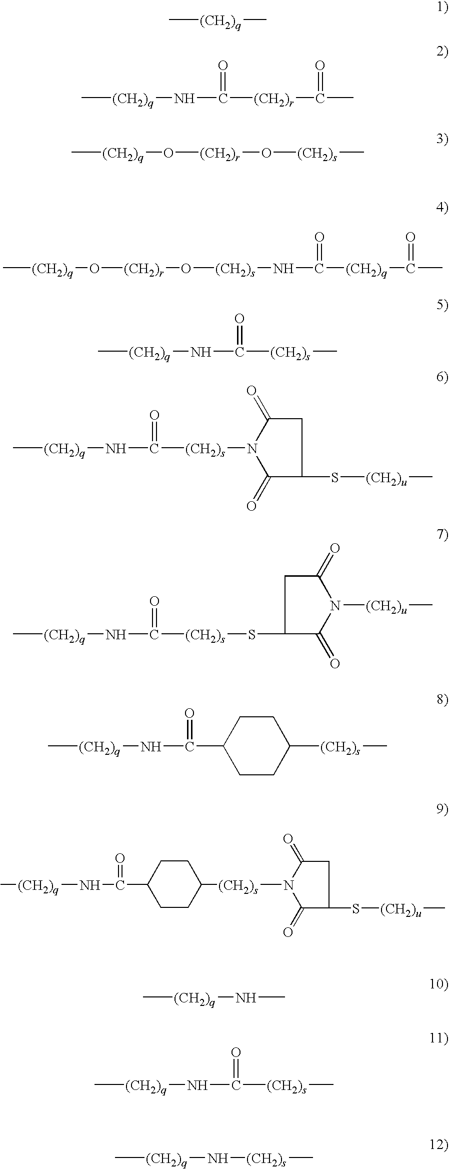
in which:
n and m are integers from 1 to 16, preferably 1 to 8;
p and r are integers from 1 to 16, preferably 1 to 5.

18. Lanthanide (III) ion complexing compound according to claim 1, wherein the reactive group Rg is chosen from the group consisting of: carboxylic acids esters, activated carboxylic acid ester, aldehydes, alkyl halides, amines, anhydrides, aryl halides, carboxylic acids, haloacetamides, halotriazines, hydrazines (including hydrazides), isocyanates, isothiocyanates, maleimides, phosphoramidites, sulfonyl halides, and thiol groups, succinimidyl ester of a carboxylic acid, or a combination thereof

19. Lanthanide (III) ion complexing compound according to claim 1, wherein the reactive group Rg is chosen from the group consisting of: 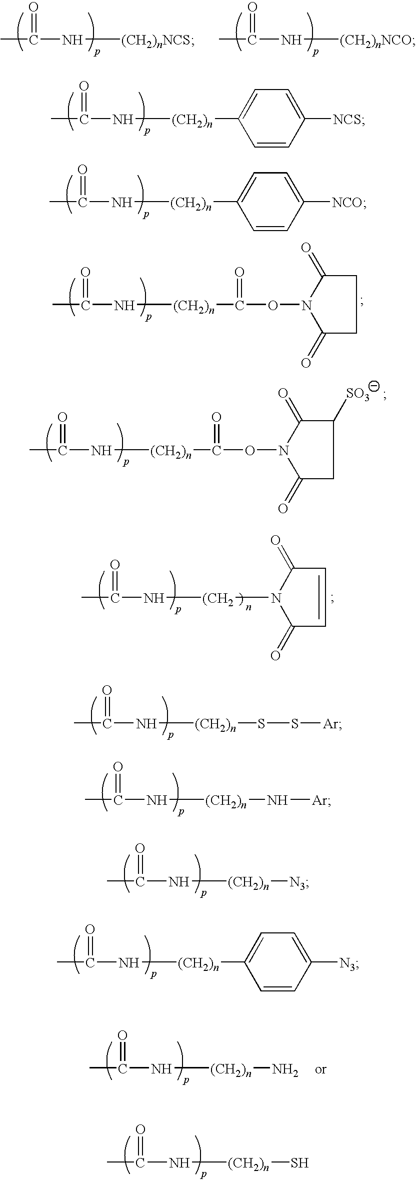
in which:
p represents 0 to 8 and n represents 0 or 1;
Ar is 5 to 6 member aryl, optionally containing 1 to 3 heteroatoms chosen from halo, N, O, S and optionally substituted by a halogen atom.

20. Lanthanide (III) ion complexing compound according to claim 1, wherein the conjugated substance Sc is biomolecule chosen from: an amino acid, a peptide, a protein, a nucleoside, a nucleotide, an oligonucleotide, a nucleic acid polymer, or a carbohydrate.

21. Lanthanide (III) ion complexing compound according to claim 20 wherein the conjugated substance Sc is a member of a specific binding pair chosen from: antigen/antibody, avidin or streptavidin/biotin, ligand/receptor, DNA strand/complementary DNA strand

22. Luminescent lanthanide (III) ion complex comprising a (III) ion complexing compound according to claim 1 and a lanthanide ion chosen from the group consisting of Tb³⁺, Eu³⁺, Sm³⁺, Dy³⁺.

23. A fluorescent label comprising a luminescent lanthanide (III) ion complex according to claim 22.

24. Sensitising derivative of formula (Ia): 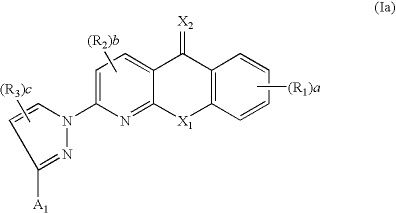
in which:
(R₁)ₐ, (R₂)_{b} and (R₃)_{c} are as defined for moiety of formula (I) in claim 1;
A₁ is hydrogen, alkyl, halogen or halogenoalkyl.

25. Sensitising derivative of formula (Ia) according to claim 24, wherein X₁═X₂═O.

26. Sensitising derivative of formula (Ia) according to claim 24, wherein a=b=c=1, R₂═R₃═H and R₁ is a (C₁-C₆) alkyl.

27. Sensitising derivative of formula (Ia) according to claim 24, wherein X1═X2═O, a=b=c=1, R₂═R₃═H and R₁ is a (C₁-C₆) alkyl.

28. Sensitising derivative of formula (Ia) according to claim 24, wherein
a═b═c═1;
R₁═H, (C₁-C₆) alkyl;
R₂═H;
R₃═CF₃; COOR₄, where R₄═H, (C₁-C₆) alkyl, aryl, CN, halo, phenyl;
X₁═X₂═O.
